(19) United States
(12) Patent Application Publication
(10) Pub. No.: US 2004/0006783 A1
(43) Pub. Date: Jan. 8, 2004

(54) Compositions and methods for modulating Rop GTPase activity in plants

(75) Inventors: Zhenbiao Yang, Riverside, CA (US); Julia Bailey-Serres, Anaheim Hills, CA (US); Airica Baxter-Burrell, Norco, CA (US); Guang Wu, San Marcos, CA (US); Vanessa Vernoud, Riverside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS THE UNIVERSITY OF CALIFORINIA, Oakland, CA (US)

(21) Appl. No.: 10/172,526

(22) Filed: Jun. 13, 2002

(51) Int. Cl. A01H 1/00; C12N 9/50; C12N 15/82; C07H 21/04; C12P 21/02; C12N 5/04

(52) U.S. Cl. 800/278; 435/69.1; 435/219; 435/320.1; 435/419; 536/23.2

(57) ABSTRACT

The present invention provides methods and compositions for regulating Rop GTPase activity in a plant.

### STATEMENT AS TO RIGHTS TO INVENTIONS MADE UNDER FEDERALLY SPONSORED RESEARCH OR DEVELOPMENT

This invention was made with Government support under Grant No. 97-35100-4191 and 01-35304-09894, awarded by the United States Department of Agriculture, and Grant No. DE-7G03-00ER15060, awarded by the Department of Energy. The government has certain rights in this invention.

### BACKGROUND OF THE INVENTION

Plants are obligate aerobes. Poor soil drainage, ice encapsulation and flooding reduce the amount of oxygen available for completion of mitochondrial electron transport chain. Flooding from monsoon rains inundate approximately 20% of low-land rice paddies throughout the world each year. Yield losses due to flash flooding are significant in China and Southeast Asia. A major goal of international rice breeders is to generate low-land cultivars that can withstand 14 days of submergence at the seedling stage. In rice growing areas with controlled irrigation practices, such as California, farmers are anxious to obtain japonica cultivars that can be pre-germinated and then seeded into flooded fields. The availability of such lines would significantly reduce the levels of herbicide applied at the pre- and post-emergence stages.

Adaptation of maize and rice seedlings to flooding involves changes in physiology and morphology during the onset of inundation. The partial submergence of seedlings or exposure to hypoxia promotes adaptations, including increased ethanolic fermentation, development of aerenchyma and lateral roots and development of a lactate efflux system in maize (Drew, Annu. Rev. Plant Physiol. Plant Mol. Biol. 48:223-250 (1977)).

Oxygen deprivation is normally detrimental during seed germination and the establishment of seedlings. However, some rice genotypes and rice weeds (e.g. some species of Echinochloa) are unusual in their ability to germinate under in the absence of oxygen (Kennedy, et al., Plant Physiol 100:1-6 (1992)). Limited oxygen availability dramatically represses root elongation but coleoptile elongation continues (Ellis et al., J Plant Physiol 154:219-230 (1999)). The ability of rice coleoptiles to elongate under low oxygen levels provides a means to outgrow submergence. Nonetheless, low-land rice seedlings are typically unable to survive complete submergence for periods longer than one week (Crawford, STUDIES IN PLANT SURVIVAL (Blackwell Press, Oxford, 1989)). Seedling death is generally thought to result from the expenditure of stored and soluble carbohydrates.

Short-term tolerance of oxygen deprivation is observed in maize and low-land rice varieties. These plants undergo metabolic adjustments in roots and coleoptiles in response to complete submergence. Oxygen deprivation promotes the consumption of stored carbohydrates, accelerated production of ATP through glycolysis and regeneration of NAD⁺ through ethanolic fermentation (Pasture effect). ATP generation under low-oxygen stress is necessary to maintain vacuolar membrane potential in order to avoid acidification of the cytosol. In rice, maize and Arabidopsis, oxygen deprivation stimulates changes in gene transcription that promotes an increase in accumulation of mRNAs that encode enzymes involved in starch and sucrose metabolism as well as pyruvate decarboxylase (PDC) and ADH. Genotypes with extremely reduced ADH levels are sensitive to oxygen deprivation (maize, Schwartz D, Amer. Naturalist 103:479-481 (1969); Arabidopsis, Jacobs et al. Biochem Genet. 26:105-122 (1988), rice, Matsumura et al. Breed Sci. 45:365-367 (1995)), confirming that ethanolic fermentation is required for flooding tolerance. However, there is no positive correlation between ADH specific activity (or ethanol production) in submerged organs and tolerance of flooding among rice cultivars (Setter et al. Ann. Bot. 74:273-279 (1994)).

Thus, it is not clear how to modify plant gene expression to engineer flood tolerance. This and other advantages are provided by the present application.

### BRIEF SUMMARY OF THE INVENTION

The present invention provides nucleic acids comprising a heterologous plant promoter operably linked to a polynucleotide encoding a RopGAP polypeptide. In some embodiments, the RopGAP polypeptide comprises a Cdc42/Rac-interactive binding (CRIB) motif and a GAP domain; the RopGAP polypeptide inactivates Rop GTPase signaling; and the heterologous promoter is expressed in a plant tissue other than pollen. In some embodiments, the RopGAP polypeptide is selected from the group consisting of Arabidopsis RopGAP1 (SEQ ID NO:2), Arabidopsis RopGAP2 (SEQ ID NO:4), Arabidopsis RopGAP3 (SEQ ID NO:6), Arabidopsis RopGAP4 (SEQ ID NO:8), Arabidopsis RopGAP5 (SEQ ID NO: 10) and Arabidopsis RopGAP6 (SEQ ID NO: 12).

In some embodiments, the heterologous promoter comprises a ROS-inducible element. In some embodiments, the heterologous promoter induces expression of the polynucleotide in the presence of less ROS than required to induce a native RopGAP promoter. In some embodiments, the heterologous promoter comprises an ADH promoter. In some embodiments, the heterologous promoter induces expression of the polynucleotide in the presence of more ROS than required to induce a native RopGAP promoter.

In some embodiments, the polynucleotide is in a sense orientation compared to the heterologous promoter. In some embodiments, the polynucleotide is in an antisense orientation compared to the heterologous promoter.

In some embodiments, the heterologous promoter is seed-specific. In some embodiments, the heterologous promoter is endosperm-specific. In some embodiments, the heterologous promoter is embryo-specific. In some embodiments, the heterologous promoter is root-specific. In some embodiments, the heterologous promoter is a senescence-specific promoter.

The present invention also provides nucleic acids comprising a heterologous plant promoter operably linked to a polynucleotide encoding a dominant negative RopGAP polypeptide. In some embodiments, the dominant negative RopGAP polypeptide comprisea an amino acid sequences at least 80% identical to SEQ ID NO: 13 and SEQ ID NO: 14. In some embodiments, the dominant negative RopGAP polypeptide is a RopGAP polypeptide lacking a GAP domain. In some embodiments, the dominant negative RopGAP polypeptide a conserved arginine residue of a RopGAP GAP domain is altered.

The present invention also provides plants comprising a heterologous promoter operably linked to a polynucleotide encoding a RopGAP polypeptide. In some embodiments, the RopGAP polypeptide comprises a Cdc42/Rac-interactive binding (CRIB) motif and a GAP domain; the RopGAP polypeptide inactivates Rop GTPase signaling; and the heterologous promoter is expressed in a plant tissue other than pollen.

In some embodiments, the plant has increased tolerance for low oxygen levels than a nontransgenic plant. In some embodiments, the heterologous promoter induces expression of the polynucleotide in the presence of more ROS than required to induce a native RopGAP promoter.

In some embodiments, the plant is a rice plant.

In some embodiments, the heterologous promoter is seed-specific. In some embodiments, the heterologous promoter is root-specific. In some embodiments, the heterologous promoter is senescence-specific.

In some embodiments, the plant has increased tolerance for reactive oxygen species levels than a nontransgenic plant. In some embodiments, the plant has increased tolerance for biotic or abiotic stresses than a nontransgenic plant. In some embodiments, the plant has delayed scenescence compared to a nontransgenic plant.

The present invention also provides plants comprising a heterologous plant promoter operably linked to a polynucleotide encoding a dominant negative RopGAP polypeptide. In some embodiments, the dominant negative RopGAP polypeptide comprisea an amino acid sequence at least 80% identical to SEQ ID NO:13 and SEQ ID NO:14. In some embodiments, the dominant negative RopGAP polypeptide is a RopGAP polypeptide lacking a GAP domain. In some embodiments, the dominant negative RopGAP polypeptide a conserved arginine residue of a RopGAP GAP domain is altered.

The present invention also provides methods of modulating negative feedback regulation of a Rop GTPase in a plant. In some embodiments, the methods comprise: introducing an expression cassette comprising a heterologous promoter operably linked to a polynucleotide encoding a RopGAP polypeptide, wherein the RopGAP polypeptide comprises a Cdc42/Rac-interactive binding (CRIB) motif and a GAP domain; the RopGAP polypeptide inactivates Rop GTPase signaling; and the heterologous promoter is expressed in a plant tissue other than pollen. In some embodiments, the methods further comprise the step of selecting a plant that has increased tolerance for reactive oxygen species levels compared to a nontransgenic plant.

The present invention also provides methods of modulating negative feedback regulation of a Rop GTPase in a plant. In some embodiments, the methods comprise: introducing into the plant an expression cassette comprising a heterologous plant promoter operably linked to a polynucleotide encoding a dominant negative RopGAP polypeptide. In some embodiments, the dominant negative RopGAP polypeptide comprisea an amino acid sequences at least 80% identical to SEQ ID NO: 13 and SEQ ID NO: 14. In some embodiments, the dominant negative RopGAP polypeptide is a RopGAP polypeptide lacking a GAP domain. In some embodiments, the dominant negative RopGAP polypeptide a conserved arginine residue of a RopGAP GAP domain is altered.

### Definitions

The phrase “nucleic acid sequence” refers to a single or double-stranded polymer of deoxyribonucleotide or ribonucleotide bases read from the 5′ to the 3′ end.

A “promoter” is defined as an array of nucleic acid control sequences that direct transcription of an operably linked nucleic acid. As used herein, a “plant promoter” is a promoter that functions in plants. Promoters include nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation.

“Tissue specific” promoters are transcriptional control elements that are active in particular cells or tissues at specific times during plant development, such as in vegetative tissues or reproductive tissues. In addition, tissue-specific promoter may drive expression of operably linked sequences in tissues other than the target tissue. Thus, as used herein a tissue-specific promoter is one that drives expression preferentially in the target tissue, but may also lead to some expression in other tissues as well.

A “ROS-inducible element” refers to a cis-acting promoter element that enables a promoter to be transcriptionally activated in response to levels of reactive oxygen species (e.g., hydrogen peroxide) levels. Exemplary ROS-inducible elements include, e.g., the antioxidant response element (called an ARE or Electrophile response element) and the anaerobic response element (also called an ARE in scientific literature, but referred to herein as AnaRE). The consensus sequence of ARE is 5′-GTGACA(A/T)(A/T)GC-3′ (Marrs, Annu. Rev. Plant Physiol. Plant Mol. Biol. 47:127-58 (1996)). AnaRE is required for activation of gene transcription in response to low oxygen stress in some promoters in monocots and dicots. An AnaRE contains two motifs: a GT-motif with a consensus of 5′-(T/C)GGTTT-3′ and two GC-motifs with the consensus 5′-GCC[G/C]C-3′. The orientation of these motifs relative to the coding sequence varies, but they are typically located between −100 and −200 nucleotides from the start of transcription. The G/T-motifs of the Arabidopsis ADH AnaRE bind to the MYB2 transcription factor in an in vitro gel-shift assay and transient expression system. See, e.g., Hoeren et al. Genetics 149:479-490 (1998). An AnaRE-like element also is found in the RopGAP4 promoter. Transcription of AnaRE-containing genes, such as ADH and RopGAP4 in Arabidopsis, is regulated via Rop GTPase signal transduction following an increase in hydrogen peroxide levels. Additional ROS-inducible elements can be identified in promoters that are induced by ROS (e.g., an ADH promoter). ROS-inducible transcripts can be identified using genechip transcriptional analyses.

A “senescence-specific” promoter refers to a promoter that initiates transcription primarily in cells going through senescence. Exemplary senescence-specific promoters include, e.g., the SAG12 promoter from Arabidopsis (see, e.g., Noh, et al., Plant Mol Biol. 41(2):181-94 (1999)).

An “ADH promoter” as used herein, refers to the polynucleotide sequence upstream of the start codon of an alcohol dehydrogenase gene from any species. Typically, the promoter will be between 200-5,000 nucleotides and will generally be between 500-2,000 nucleotides. Exemplary ADH promoters include the ADH promoters from maize (see, e.g., Dennis, et al., Nucleic Acids Res. 12(9):3983-4000 (1984)), pea (Llewellyn, et al., J Mol Biol. 195(1):115-23 (1987)) and Arabidopsis (see, e.g., McKendree, et al., Plant Mol Biol. 19(5):859-62 (1992); Dolferus, et al., Plant Physiol. 105(4):1075-87 (1994)).

A “native” promoter or gene sequence refers to a nucleotide sequence found in a nontransgenic plant that has not been submitted to mutagenesis by humans.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is different from any naturally-occurring allelic variants.

A polynucleotide “exogenous to” an individual plant is a polynucleotide which is introduced into the plant, or a predecessor generation of the plant, by any means other than by a sexual cross. Examples of means by which this can be accomplished are described below, and include Agrobacterium-mediated transformation, biolistic methods, electroporation, in planta techniques, and the like.

An “expression cassette” refers to a nucleic acid construct, which when introduced into a host cell, results in transcription and/or translation of an RNA or polypeptide, respectively. Antisense or sense constructs that are not or cannot be translated are expressly included by this definition.

“Tolerance of low oxygen levels” refers to the ability of a plant survive low oxygen conditions, such as flooding, for a period of time and to recover once the low oxygen conditions have passed. “Increased tolerance,” in this context, refers to an ability of plant to survive low oxygen conditions for a longer period of time, or to recover more quickly, than a control plant. Where a transgenic plant is tested for tolerance, a control plant could be a non-transgenic plant from the same plant line.

A “RopGAP nucleic acid” or “RopGAP polynucleotide sequence” of the invention is a subsequence or full length polynucleotide sequence of a gene which encodes a polypeptide comprising a Cdc42/Rac-interactive binding (CRIB) motif and a GAP domain. The present invention provides polypeptides that comprise CRIB motifs and GAP domains substantially identical to those exemplified herein. Exemplary CRIB motifs and GAP domains are at least 70% identical to those described here (e.g., SEQ ID NOs: 11 and 12), and in some embodiments are at least 75%, 80%, 85%, 90%, or 95% identical. RopGAPs stimulates Rop GTPase activity, i.e. GTP hydrolysis.

“Cdc42/Rac-interactive binding motifs” or “CRIB motifs” refers to protein motifs that interact with G-proteins. Two invariant histidines in CRIB motifs are involved in interaction with G-proteins (Abdul-Manan et al, Nature 399:379-383 (1999); Mott et al., Nature 399:384-388 (1999)). CRIB motif are present in plant RopGAP proteins (Wu et al., Plant Physiol. 124:1625-1636 (2000)). For example, the CRIB motif of RopGAP4 is: M₉₁EIGWPTDVRHVAHVTFDRFHGFLGLP₁₁₇ (SEQ ID NO:11) (the bold residues are variant among putative plant RopGAPs). The CRIB motif in Arabidopsis RopGAP1 is M₁₅EIGWPTNVRH₁₂₅VAH₁₂₈VTFDRNGGFLGLP₁₄₁. Thus, a consensus motif for RopGAP CRIB motifs is IGXXTXVXHXXHVTFDRXXGFXGLP. Mutation of either H₁₂₅ or H₁₂₈ to Y in RopGAP 1 causes a dramatic decrease in the activation of hydrolysis of Rop1-GTP. A slightly more severe reduction in GTPase activity is observed with a RopGAP 1 mutant lacking the N-terminus and CRIB motif (residues 1 to 143 of RopGAP 1) (Wu et al., Plant Physiol. 124:1625-1636 (2000)). Without intending to limit the invention to a particular mechanism of action, it is believed that the CRIB motif of RopGAP stimulates GTPase activity by promoting the formation of the transitional state in GTP hydrolysis.

“GAP domains” of GTPase activating proteins are characterized by one to three sub domains or motifs, each of which contains one or more invariant arginine residues. See, e.g., Rittinger et al., Nature 389: 758-762 (1997); Leonard et al., J Biol. Chem. 273:16210-16215 (1998); and Scheffzek et al, Trends Biochem. 23:257-272 (1998). Motif I forms a finger with the universally conserved arginine residue that is essential for G-protein interaction. Crystalographic data suggest that motif II stabilizes the arginine finger formed by motif I. The GAP domain of RopGAP 1 was coarsely defined by deletion mutations to exist between residue 143 to 352 (the corresponding region of RopGAP4 spans from residue 119 to 336) (Wu et al, Plant Physiol. 124:1625-1636 (2000)). This region is necessary for stimulation of Rop1 GTP hydrolysis. R₁₇₈ of RopGAP4 corresponds to the invariant arginine found in Motif I of GTPase activating proteins and is similarly preceded by an aromatic residue (Scheffzek et al., Trends Biochem. 23:257-272 (1998)). In some cases, additional arginine residues are conserved within the GAP domain of RopGAPs. These arginines correspond to R₂₁₆, R₂₇₇ and R₃₁₈ of RopGAP4.

Plant RopGAP proteins are unique in the novel combination of a CRIB motif and a GAP domain in the central portion of the protein. RopGAPs show high amino acid sequence conservation within this CRIB+GAP region and divergence at the amino and carboxyl termini. A 220 amino acid region, from M₉₁ to L₃₁₀ comprises CRIB and GAP regions of RopGAP4, follows:

M₉₁EIGVPTDVR HVAHVTFDRF HGFLGLPVEF EPEVPRRAPS
ASATVFGVST ESMQLSYDTR GNIVPTILLM MQSHLYSRGG
LRVEGIFR₁₇₈IN GENGQEEYIR EELNKGIIPD NIDVHCLASL
IKAWFR₂₁₆ELPS GVLDSLSPEQ VMESESEDEC VELVRLLPST
EASLLDWAIN LMADVVEMEQ LNKMNAR₂₇₇NIA MVFAPNMTQM
LDPLTALMYA VQVMNFLKTL₃₁₀.

Sequence identity in this region is greater than 50% between putative RopGAPs of eudicots and monocots. The RopGAP4 region comprising L₂₇₁NKMNAR₂₇₇NIAMVFAPNMTQMLDPLTALMYAVQVMNFLKTL₃₁₀ (SEQ ID NO: 14) shows greater than 80% amino acid sequence identity between putative RopGAPs of higher plants. Thus, the present invention provides polypeptides that comprise a sequence substantially identical to this region. In some embodiments, the RopGAPs of the invention comprise sequences at least substantially identical identical to the above-listed 220 amino acid region, from M₉₁ to L₃₁₀ of RopGAP4, and in some embodiments are at least 70%, 75%, 80%, 85%, 90%, or 95% identical.

Exemplary polynucleotides of the invention include those substantially identical to, e.g., the Arabidopsis RopGAPs1-5 (see, e.g., Wu et al, Plant Physiol. 124:1625-1636 (2000)) and RopGAP6 (At2g27440), as well as Lotus RopGAPs described in Borg et al., FEBS Lett. 453:341-345 (1999); erratum Borg et al., FEBS Lett. 458:82 (1999). RopGAP polynucleotides can typically hybridize under defined conditions to the exemplified nucleic acids or PCR products derived from them. An RopGAP polynucleotide is typically at least about 50 to about 5,000 nucleotides in length, and is sometimes between 500 to 3000 base pairs in length.

In the case of both expression of transgenes and inhibition of endogenous genes (e.g., by antisense, or sense suppression) one of skill will recognize that the inserted polynucleotide sequence need not be identical, but may be only “substantially identical” to a sequence of the gene from which it was derived. As explained below, these substantially identical variants are specifically covered by the term RopGAP nucleic acid or RopGAP polynucleotide.

In the case where the inserted polynucleotide sequence is transcribed and translated to produce a functional polypeptide, one of skill will recognize that because of codon degeneracy a number of polynucleotide sequences will encode the same polypeptide. These variants are specifically covered by the terms “RopGAP nucleic acid”. In addition, the term specifically includes those sequences substantially identical (determined as described below) with a RopGAP polynucleotide sequence disclosed here and that encode polypeptides that are either mutants of wild type RopGAP polypeptides or retain the function of the RopGAP polypeptide (e.g., resulting from conservative substitutions of amino acids in the RopGAP polypeptide). In addition, variants can be those that encode dominant negative mutants as described below.

A “dominant negative RopGAP” refers to a polypeptide variant of a native RopGAP sequence whose expression interferes with or otherwise counteracts native RopGAP activity. Dominant negative RopGAP mutants can include a fragment of a RopGAP polypeptide sequence with at least one mutation. Exemplary mutations include, e.g., RopGAP polypeptide where at least one a conserved arginine residue in a RopGAP GAP domain is replaced with another amino acid. Alternatively, a dominant negative mutant can lack one of a GAP domain, a CRIB motif, or other domains or motifs. In some embodiments, the dominant negative RopGAPs comprise a polypeptide at least 50%, 60%, 70%, 80%, or 90% identical to SEQ ID NOs: 2, 4, 6, 8, 10, or 12. In some embodiments, the dominant negative mutant RopGAPs comprise an amino acid sequence that is at least 50%, 60%, 70%, 80%, or 90% identical to SEQ ID NO:13 and/or SEQ ID NO: 14.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The term “substantial identity” of polynucleotide sequences means that a polynucleotide comprises a sequence that has at least 25% sequence identity. Alternatively, percent identity can be any integer from at least 25% to 100% (e.g., at least 25%, 26%, 27%, 28%, . . . ,70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%). More preferred embodiments include at least: 25%, 30%, 35%, 40%, 45%, 50%, 55%, 60%, 65%, 70%, 75%, 80%, 85%, 90%, 95%, or 99% compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%. Preferred percent identity of polypeptides can be any integer from at least 40% to 100% (e.g., at least 40%,41%, 42%, 43%, 70%, 0.71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%). More preferred embodiments include at least 60%, 65%, 70%, 75%, 80%, 85%, 90%, 95%, or 99%. The present invention provides polypeptides (and polynucleotides encoding such polypeptides) substantially identical to the sequences exemplified herein (e.g., Arabidopsis RopGAPs1-6) as well as polypeptide comprising sequences substantially identical to the RopGAP4 CRIB motif and GAP domain. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Unless other wise indicated, the comparison window extends the entire length of a reference sequence. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

One example of a useful algorithm that is suitable for determining percent sequence identity and sequence similarity is the BLAST algorithm, which is described in Altschul et al., J. Mol Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=-4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (O);

4) Arginine (R), Lysine (K);

5) Isoleucine (I), Leucine (L), Methionine (M), Valine (V); and

6) Phenylalanine (F), Tyrosine (Y), Tryptophan (W). (see, e.g., Creighton, Proteins (1984)).

An indication that two nucleic acid sequences or polypeptides are substantially identical is that the polypeptide encoded by the first nucleic acid is immunologically cross reactive with the antibodies raised against the polypeptide encoded by the second nucleic acid. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, highly stringent conditions are selected to be about 5-10° C. lower than the thermal melting point (Tₘ) for the specific sequence at a defined ionic strength pH. Low stringency conditions are generally selected to be about 15-30° C. below the Tₘ. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 time background hybridization.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cased, the nucleic acids typically hybridize under moderately stringent hybridization conditions.

In the present invention, genomic DNA or cDNA comprising RopGAP nucleic acids of the invention can often be identified in standard Southern blots under stringent conditions using the nucleic acid sequences disclosed here. For the purposes of this disclosure, suitable stringent conditions for such hybridizations are those which include a hybridization in a buffer of 40% formamide, 1 M NaCl, 1% SDS at 37° C., and at least one wash in 0.2×SSC at a temperature of at least about 50° C., usually about 55° C. to about 60° C., for 20 minutes, or equivalent conditions. A positive hybridization is at least twice background. Those of ordinary skill will readily recognize that alternative hybridization and wash conditions can be utilized to provide conditions of similar stringency.

A further indication that two polynucleotides are substantially identical is if the reference sequence, amplified by a pair of oligonucleotide primers, can then be used as a probe under stringent hybridization conditions to isolate the test sequence from a cDNA or genomic library, or to identify the test sequence in, e.g., a northern or Southern blot.

### BRIEF DESCRIPTION OF THE DRAWINGS

FIG. 1A illustrates the site and orientation of DsG insertion within the CRIB motif of the first exon of Arabidopsis RopGAP4 in the mutant ropgap4-1. FIG. 1B illustrates RT-PCR detection of ADH, RopGAP4, and actin (ACT2) mRNA in WT, ropgap4-1, CA-rop2 and DN-rop2 seedlings following O₂ deprivation.

FIG. 2A illustrates ADH specific activity in Arabidopsis seedlings (7 day old) following oxyegn deprivation in the absence or presence of 30 μM DPI, FIG. 2B illustrates results of a Rop-RIC1 interaction assay on extracts from WT and ropgap4-1 seedlings following oxyegn deprivation. Immunoblot detection of levels of total Rop (Rop-GTP and Rop-GDP) in crude extracts or Rop-GTP obtained by pull-down through interaction with RIC1-maltose binding protein. Data are representative of three independent experiments. FIG. 2C illustrates H₂O₂ levels following oxyegn deprivation. In FIGS. 2A and 2C, values are the mean±SE of three independent experiments. An asterisk indicates a significant difference from WT at the same time point (P<0.01, Student's t test).

FIG. 3A illustrates ADH specific activity in seedlings treated with caffeine and/or DPI for 24 h. FIG. 3B illustrates H₂O₂ levels in seedlings analyzed in A. Values are the mean±SE of three independent experiments. An asterisk indicates a significant difference from the maximal level detected following oxyegn deprivation (P<0.01, Student's t test). FIG. 3C illustrates GUS specific activity in ropgap4-1 seedlings following oxyegn deprivation, caffeine and DPI treatment. Values are the mean±SE of three independent experiments.

FIGS. 4 illustrates ADH specific activity in WT (A) and GUS specific activity in ropgap4-1 seedlings (B) treated with glucose and glucose oxidase for up to 3 hours.

FIG. 5 provides a schematic illustration of the Rop GTPase signal transduction and negative feedback regulation of Rop GTPase signaling by the GTPase activating protein, RopGAP.

### DETAILED DESCRIPTION OF THE INVENTION

I. Introduction

The present invention provides compositions and methods for regulating Rop GTPase signal transduction in plants. A wide variety of plant phenotypes are regulated by Rop GTPases. These include, abiotic and biotic stress responses, cell morphology, cytoskeleton dynamics, secondary cell wall development, pollen tube growth, embryo development, seed dormancy, seedling development, lateral root initiation, shoot apical dominance, shoot growth, lateral organ formation, phyllotaxis, lateral organ orientation and responses to auxin, abscisic acid and brassinolide. These phenotypes can therefore be modulated by manipulating the expression of a RopGAP polypeptide, which down regulates Rop GTPase signaling, thereby modulating Rop GTPase activity. Modulation of RopGAP expression can be achieved, for example, by ectopic expression of a RopGAP polypeptide, by up- or down-regulating endogenous RopGAP expression, by manipulation of the activity of a RopGAP, or by manipulating the timing and/or the location of expression of a RopGAP, e.g., by using an inducible and/or tissue-specific promoter and/or inducing more rapid or slower RopGAP expression. Thus, Rop GTPase signaling regulates multiple developmental processes and environmental responses.

The compositions and methods of the invention are useful, for example, for regulating ROS-mediated plant phenotypes, e.g., the biotic and abiotic response and developmental processes described above. These phenotypes can be regulated by manipulating the timing and level of expression of RopGAPs in a plant. In many embodiments of the invention, expression of RopGAPs in a specific tissue, subcellular compartment, or under certain conditions, allows for precise regulation of a desired phenotype.

II. Phenotypic Manipulation of Plants by Modulating the Level and Timing of RopGAP Expression and ROPGAP Activity

Rop GTPase signal transduction is finely regulated by RopGAP expression in a feedback loop that modulates and maintains activity of Rop GTPase. RopGAPs act as negative regulators of Rop GTPase signaling. A signal such as oxygen deprivation activates Rop GTPase, which in turn stimulates NADPH oxidase activity, resulting in an elevation in reactive oxygen species (ROS), including hydrogen peroxide. As second messengers, ROS regulates the expression of genes such as ADH. Levels of ROS are elevated in the response to many abiotic stimuli (e.g., excessive light, chilling, osmotic shock, drought, UV irradiation, oxygen deprivation, etc.) and biotic stimuli (elicitors, pathogen infection and wounding). In addition, ROS are involved in modulation of developmental programs (e.g., differentiation of secondary cell walls, programmed cell death, senescence, etc.) and hormonal responses (e.g. auxin and abscisic acid) and the hypersensitive response to pathogens. Variation in the level and duration of ROS production will have consequences on phenotype. However, excessive prolonged accumulation of ROS causes plant cell death. Therefore, it is useful to balance the activation of Rop GTPase signaling (and subsequently the production of beneficial ROS and the attainment of a desired plant phenotype) with RopGAP-mediated Rop inactivation that prevents the accumulation of the toxic levels of ROS. Thus, phenotypes under ROP GTPase regulation can be controlled by manipulation of the regulation of expression or activity of RopGAP.

Rop GTPase regulates ROS accumulation and ADH expression. Endogenous RopGAP expression is controlled, in part, by a reactive oxygen species (ROS)-inducible promoter. Since hydrogen peroxide is produced upon Rop GTPase activation, Rop GTPase activates its own inhibitor. Thus, controlling the level of Rop GTPase activity can be used to regulate ROS accumulation, thereby preventing significant ROS-induced cellular damage and allowing for manipulation of Rop GTPase determined phenotypes. ROS act in a variety of developmental and inducible systems. In some contexts, ROS act to stimulate desirable phenotypes. For example, Rop and RopGAP4 interact to regulate Rop GTPase signaling involved in low oxygen tolerance. For rice and other crop plants where it is desirable to germinate seed under water, submergence tolerance requires both the survival of temporary O₂ deprivation as well as recovery of growth upon the return to non-flooded conditions. Submergence tolerance of young seedlings may require the conservative consumption of carbohydrate resources plus control the accumulation of toxic substances under stress conditions and during recovery. Submergence tolerance can be achieved by modulating RopGAP expression under low oxygen conditions.

Tolerance of low oxygen conditions is triggered in part by H₂O₂ accumulation, which induces expression of alcohol dehydrogenase (ADH). Accumulation of ADH without an overabundance of ROS, results in a significant increase in low oxygen tolerance. The timing and level of ROS production controls low oxygen tolerance. Therefore, modulating Rop GTPase signaling could be used to increase tolerance. In a plant or cell-type in which insufficient ADH is produced, reducing or delaying, but not eliminating, RopGAP expression increases low oxygen tolerance. In a plant or cell-type in which ADH or ROS is over-produced, increasing or more rapidly stimulating RopGAP expression increases low oxygen tolerance.

Similarly, manipulation of Rop GTPase signalling also affects chilling tolerance. In this case, a more rapid down regulation of Rop-signaling (i.e., caused by a more rapid RopGAP expression) enhances chilling tolerance.

ROS accumulation is also implicated in plant defense response to pathogens. In this case, ROS production initially stimulates the hypersensitive response but ultimately results in cell death around the site of infection. Thus, reduction of RopGAP expression in cells participating in a defense response can be used to stimulate the hypersensitive response and plant resistance. Delaying RopGAP expression can promote cell lesions but enhances the hypersensitive response to restrict the spread of pathogens to uninfected cells. Alternatively, a more rapid induction of RopGAP or higher expression levels of RopGAP increases pathogen spread within plant tissues. Defense-specific or disease-specific promoters can be linked to RopGAP constructs (i.e., antisense or dominant negative embodiments) to reduce endogenous RopGAP expression in cells responding to pathogen invasion, thereby enhancing plant resistance to pathogens.

ROS accumulation is also implicated in plant scenescence. Rop GTPase signaling is responsible, at least in part, for this ROS. Thus, increasing RopGAP expression delays senescence. Promoter elements that induce expression in senescent tissues can be used to express RopGAP in senescent tissue.

Inactivation of Rop signaling initiates abscisic acid-induced closure of leaf stomata. Therefore, drought tolerance can be engineered in a plant by increasing expression of RopGAP in stomata under conditions when the plant is exposed to drought conditions. Thus, expression of RopGAP under the control of a stomata-specific promoter, an ABA-inducible promoter, or early drought-inducible promoter is useful for creating drought resistant plants.

Rop activation also stimulates secondary wall differentiation (i.e. cellulose synthesis and/or deposition). See, e.g., Potikha et al., Plant Physiology 849-858 (1999). Thus modulation of RopGAP expression can be used to modulate secondary wall differentiation. In some aspects, cotton fiber production and quality can be manipulated by modulating RopGAP expression in cotton fibers.

Rop activation is involved in root hair formation. For example, down regulation of endogenous RopGAP activity or expression (e.g., by expression of a dominant negative RopGAP or antisense or sense suppression) increases root hair length and root hair number, resulting in increased nutrient and water uptake from the soil.

Rop activation is involved in lateral root formation. Expression of a dominant negative RopGAP or inhibition of endogenous RopGAP expression increases production of lateral roots, resulting in increased nutrient and water uptake from the soil. Thus, in some embodiments, a root-specific promoter is operably linked to a RopGAP construct ito inhibit RopGAP expression.

The Rop GTPase/RopGAP system can be manipulated by changing the amount of RopGAP that accumulates in a cell. For example, RopGAP expression can be made more sensitive to ROS levels by introducing into a plant an expression cassette comprising a heterologous promoter operably linked to a RopGAP-encoding polynucleotide. The heterologous promoter can induce gene expression under lower levels of ROS than required for expression from the endogenous RopGAP promoter, thereby producing more RopGAP and resulting in a plant that will down regulate Rop GTPase much sooner (i.e., at lower ROS levels) than a native plant. For example, both the timing of RopGAP expression and level of RopGAP produced can be modulated to improve low oxygen tolerance.

Alternatively, expression of RopGAP can be made less sensitive to ROS in several ways. In some embodiments, sense or antisense constructs are used to prevent or reduce expression of an endogenous RopGAP transcript. Alternatively, expression of a dominant negative RopGAP mutant can be regulated to inhibit endogenous RopGAP activity.

In addition to ROS-inducible promoter elements, it can also be desirable to regulate RopGAP expression in a tissue-specific or developmental-specific fashion. For example, to improve low oxygen tolerance of seed and/or seedlings, specific expression of sense, antisense or dominant negative mutant RopGAP polynucleotides in the seed and/or root expression can be used.

In some embodiments, RopGAP can be expressed in specific subcellular locations, such as in an organelle (e.g. chloroplast, mitochondrion, cell nucleus) or to a membrane.

Those of skill in the art will recognize that promoter elements can be combined to construct promoters that initiate expression in novel ways. For example, a tissue-specific promoter or other promoter element can be combined with an inducible promoter to create an inducible, tissue-specific promoter. The present invention provides for any possible combination of promoter elements provided herein.

III. Nucleic Acids of the Invention

The present invention involves Rop GTPase and RopGAP polynucleotides. Polynucleotides and polypeptides are not limited to the sequences disclosed herein but include fragments and variants thereof. Those of skill in the art will recognize that conservative amino acid substitutions, as well as amino acid additions or deletions, may not result in any change in biological activity. For normal function, Rop GAP polypeptides contain a CRIB motif and a GAP domain. Thus, alterations in amino acids outside of these motifs are most likely to maintain activity.

RopGAP polynucleotides typically encode RopGAP polypeptides. Exemplary RopGAP polypeptides include, e.g., Arabidopsis RopGAPs 1-5 (see, e.g., Wu et al., Plant Physiol. 124:1625-1636 (2000)) and RopGAP6 (At2 g27440), as well as orthologous sequences from plants such as rice, cotton, tomato, wheat, maize, tobacco, and the like. Other exemplary sequences include RopGAP sequences from, e.g., lotus (Borg et al., FEBS Lett. 453:341-345 (1999); erratum Borg et al, FEBS Lett. 458:82 (1999)).

RopGAP polynucleotides also encompass polynucleotides encoding dominant negative mutations, i.e., polypeptides that prevent signal transduction or that otherwise interfere with native protein activity. Exemplary dominant negative mutants of RopGAP include, e.g., a non-conservative mutation of an invariant arginine residue within the GAP domain.

Generally, the nomenclature and the laboratory procedures in recombinant DNA technology described herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al., Molecular Cloning—A Laboratory Manual, Cold Spring Harbor Laboratory, Cold Spring Harbor, N.Y., (1989).

The isolation of nucleic acids of the invention may be accomplished by a number of techniques. For instance, oligonucleotide probes based on the sequences disclosed here can be used to identify the desired gene in a cDNA or genomic DNA library. To construct genomic libraries, large segments of genomic DNA are generated by random fragmentation, e.g. using restriction endonucleases, and are ligated with vector DNA to form concatemers that can be packaged into the appropriate vector. To prepare a cDNA library, mRNA is isolated from the desired organ, such as ovules, and a cDNA library which contains the RopGAP gene transcript is prepared from the mRNA. Alternatively, cDNA may be prepared from mRNA extracted from other tissues in which genes or homologs are expressed.

The cDNA or genomic library can then be screened using a probe based upon the sequence of a gene sequence disclosed here. Probes may be used to hybridize with genomic DNA or cDNA sequences to isolate homologous genes in the same or different plant species. Alternatively, antibodies raised against a polypeptide of the invention can be used to screen an mRNA expression library.

Alternatively, the nucleic acids of interest can be amplified from nucleic acid samples using amplification techniques. For instance, polymerase chain reaction (PCR) technology can be used to amplify polynucleotide sequences of the invention directly from genomic DNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. For a general overview of PCR see PCR Protocols: A Guide to Methods and Applications (Innis, M, Gelfand, D., Sninsky, J. and White, T., eds.), Academic Press, San Diego (1990).

Appropriate primers and probes for identifying sequences of the invention from plant tissues are generated from comparisons of the sequences provided here with other related genes. Using these techniques, one of skill can identify conserved regions in the nucleic acids disclosed here to prepare the appropriate primer and probe sequences. Primers that specifically hybridize to conserved regions (e.g., the CRIB and GAP motifs in RopGAPs) in sequences of the invention can be used to amplify sequences from widely divergent plant species.

Standard nucleic acid hybridization techniques using the conditions disclosed above can then be used to identify full-length cDNA or genomic clones.

RopGAP sequences can be tested for activity by any method known to those of skill in the art. For example, RopGAP activity can be measured by determining the ability of the encoded RopGAP to activate the conversion of Rop-GTP to Rop-GDP in Rop GTPases. See, e.g., Wu, et al., Plant Physiol. 124:1625-1636 (2000); Li et al., J. Biol. Chem. 272::32830-32835 (1997). Briefly, GTP hydrolysis is monitored for phosphatase release (e.g., using an Enz-check™ Phosphatase Assay Kit, Molecular Probes, Eugene, Oreg.) from GTP bound to an active Rop polypeptide. The phosphatase assay is performed in the presence of GTP and 2-amino-6-mercapto-7-methylpurine ribonucleoside, purine nucleotide phosphorylase and a buffer. The solution is monitored at A360 in a UV/VIS spectrometer. Once multiple turnover has reached equilibrium, a MgCl₂ solution containing RopGAP is added and the reaction is monitored by absorbance.

The polynucleotide sequence of the invention include polynucleotides altered to coincide with the codon usage of a particular host. For example, the codon usage of a monocot plant can be used to derive a polynucleotide that encodes a polypeptide of the invention and comprises preferred monocot codons. The frequency of preferred codon usage exhibited by a host cell can be calculated by averaging frequency of preferred codon usage in a large number of genes expressed by the host cell. This analysis is preferably limited to genes that are highly expressed by the host cell. U.S. Pat. No. 5,824,864, for example, provides the frequency of codon usage by highly expressed genes exhibited by dicotyledonous plants and monocotyledonous plants. Polypeptides can also be expressed in bacteria or other microorganisms and thus, codon usage can be optimized for the particular microorganism of interest.

When synthesizing a gene for improved expression in a host cell, it is desirable to design the gene such that its frequency of codon usage approaches the frequency of preferred codon usage of the host cell. The percent deviation of the frequency of preferred codon usage for a synthetic gene from that employed by a host cell is calculated first by determining the percent deviation of the frequency of usage of a single. codon from that of the host cell followed by obtaining the average deviation over all codons.

IV. Modulating Rop GAP Activity or Gene Expression

Since RopGAP genes are involved in a variety of developmental and inducible pathways, inhibition of endogenous RopGAP activity or gene expression is useful in a number of contexts. For instance, an increase in RopGAP expression can be used for increasing tolerance of low oxygen conditions, delaying senescence and controlling defense responses. In some embodiments, RopGAP expression is only reduced, not eliminated. Reduction of RopGAP expression allows for increased, but controlled, Rop GTPase activity. Alternatively, the timing of RopGAP expression can be modulated. For example, by use of a more hydrogen peroxide sensitive promoter operably linked to a RopGAP polynucleotide, expression can occur earlier, but at similar levels, than in wild type plants. Thus, the invention provides for temporal inhibition of expression, for example by use of a hydrogen peroxide-sensitive promoter driving an antisense RNA or RNAi construct.

One of skill will recognize that a number of methods can be used to modulate RopGAP activity or gene expression. RopGAP activity can be modulated in the plant cell at the gene, transcriptional, posttranscriptional, translational, or posttranslational, levels. Techniques for modulating RopGAP activity at each of these levels are generally well known to one of skill and are discussed briefly below.

Methods for introducing genetic mutations into plant genes are well known. For instance, seeds or other plant material can be treated with a mutagenic chemical substance, according to standard techniques. Such chemical substances include, but are not limited to, the following: diethyl sulfate, ethylene imine, ethyl methanesulfonate and N-nitroso-N-ethylurea. Alternatively, ionizing radiation from sources such as, for example, X-rays or gamma rays can be used.

Alternatively, homologous recombination can be used to induce targeted gene disruptions by specifically deleting or altering the RopGAP gene in vivo (see, generally, Grewal and Klar, Genetics 146: 1221-1238 (1997) and Xu et al., Genes Dev. 10:2411-2422 (1996)). Homologous recombination has been demonstrated in plants (Puchta et al., Experientia 50:277-284 (1994), Swoboda et al., EMBO J. 13:484-489 (1994); Offringa et al., Proc. Natl. Acad. Sci. USA 90: 7346-7350 (1993); Kempin et al. Nature 389:802-803 (1997); Puchta, Plant Mol Biol. 48(1-2): 173-82 (2002).

In applying homologous recombination technology to the genes of the invention, mutations in selected portions of a RopGAP gene sequences (including 5′ upstream, 3′ downstream, and intragenic regions) such as those disclosed here are made in vitro and then introduced into the desired plant using standard techniques. Alternatively, the promoter region of a RopGAP gene is altered using recombination to produce a less (or more) sensitive expression to ROS signaling, oxygen deprivation, or specific transcription factors. Since the efficiency of homologous recombination is known to be dependent on the vectors used, use of dicistronic gene targeting vectors as described by Mountford et al. Proc. Natl. Acad. Sci. USA 91:4303-4307 (1994); and Vaulont et al. Transgenic Res. 4:247-255 (1995) are conveniently used to increase the efficiency of selecting for altered RopGAP gene expression in transgenic plants. The mutated gene will interact with the target wild-type gene in such a way that homologous recombination and targeted replacement of the wild-type gene will occur in transgenic plant cells, resulting in suppression of RopGAP activity.

Alternatively, oligonucleotides composed of a contiguous stretch of RNA and DNA residues in a duplex conformation with double hairpin caps on the ends can be used. The RNA/DNA sequence is designed to align with the sequence of the target RopGAP gene and to contain the desired nucleotide change. Introduction of the chimeric oligonucleotide on an extrachromosomal T-DNA plasmid results in efficient and specific RopGAP gene conversion directed by chimeric molecules in a small number of transformed plant cells. This method is described in Cole-Strauss et al. Science 273:1386-1389 (1996) and Yoon et al. Proc. Natl. Acad. Sci. USA 93:2071-2076 (1996).

Gene expression can be inactivated using recombinant DNA techniques by transforming plant cells with constructs comprising transposons or T-DNA sequences. RopGAP mutants prepared by these methods are identified according to standard techniques. For instance, mutants can be detected by PCR or by detecting the presence or absence of RopGAP mRNA, e.g., by northern blots or reverse transcriptase PCR (RT-PCR).

The isolated nucleic acid sequences prepared as described herein, can also be used in a number of techniques to control endogenous RopGAP gene expression at various levels. Subsequences from the sequences disclosed here can be used to control, transcription, RNA accumulation, translation, and the like.

A number of methods can be used to inhibit gene expression in plants. For instance, antisense technology can be conveniently used. To accomplish this, a nucleic acid segment from the desired gene is cloned and operably linked to a promoter such that the antisense strand of RNA will be transcribed. The construct is then transformed into plants and the antisense strand of RNA is produced. In plant cells, it has been suggested that antisense suppression can act at all levels of gene regulation including suppression of RNA translation (see, Bourque Plant Sci. (Limerick) 105:125-149 (1995); Pantopoulos In Progress in Nucleic Acid Research and Molecular Biology, Vol. 48. Cohn, W. E. and K. Moldave (Ed.). Academic Press, Inc.: San Diego, Calif., USA; London, England, UK. p. 181-238; Heiser et al. Plant Sci. (Shannon) 127:61-69 (1997)) and by preventing the accumulation of mRNA which encodes the protein of interest, (see, Baulcombe Plant Mol. Bio. 32:79-88 (1996); Prins and Goldbach Arch. Virol. 141:2259-2276 (1996); Metzlaffetal. Cell 88:845-854 (1997), Sheehy et al., Proc. Nat. Acad. Sci. USA, 85:8805-8809 (1988), and Hiatt et al., U.S. Pat. No. 4,801,340).

The nucleic acid segment to be introduced generally will be substantially identical to at least a portion of the endogenous RopGAP gene or genes to be repressed. The sequence, however, need not be perfectly identical to inhibit expression. The vectors of the present invention can be designed such that the inhibitory effect applies to other genes within a family of genes exhibiting homology or substantial homology to the target gene.

For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of about 500 to about 3000 nucleotides is especially preferred.

A number of gene regions can be targeted to suppress RopGAP gene expression. The targets can include, for instance, the coding regions, introns, sequences from exon/intron junctions, 5′ or 3′ untranslated regions, and the like. In some embodiments, the constructs can be designed to eliminate the ability of regulatory proteins to bind to RopGAP gene sequences that are required for its cell- and/or tissue-specific expression. Such transcriptional regulatory sequences can be located either 5′-, 3′-, or within the coding region of the gene and can be either promote (positive regulatory element) or repress (negative regulatory element) gene transcription. These sequences can be identified using standard deletion analysis, well known to those of skill in the art. Once the sequences are identified, an antisense construct targeting these sequences is introduced into plants to control gene transcription in particular tissue, for instance, in developing ovules and/or seed. In one embodiment, transgenic plants are selected for RopGAP activity that is reduced but not eliminated. For example, an antisense RopGAP construct could be driven by a native RopGAP promoter, a low-oxygen inducible promoter (i.e. ADH or GAPC) or a senescence-inducible promoter.

Oligonucleotide-based triple-helix formation can be used to disrupt RopGAP gene expression. Triplex DNA can inhibit DNA transcription and replication, generate site-specific mutations, cleave DNA, and induce homologous recombination (see, e.g., Havre and Glazer J. Virology 67:7324-7331 (1993); Scanlon et al. FASEB J. 9:1288-1296 (1995); Giovannangeli et al. Biochemistry 35:10539-10548 (1996); Chan and Glazer J. Mol. Medicine (Berlin) 75:267-282 (1997)). Triple helix DNAs can be used to target the same sequences identified for antisense regulation.

Catalytic RNA molecules or ribozymes can also be used to inhibit expression of RopGAP genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs. Thus, ribozymes can be used to target the same sequences identified for antisense regulation.

A number of classes of ribozymes have been identified. One class of ribozymes is derived from a number of small circular RNAs that are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described in Zhao and Pick Nature 365:448-451 (1993); Eastham and Ahlering J. Urology 156:1186-1188 (1996); Sokol and Murray Transgenic Res. 5:363-371 (1996); Sun et al. Mol. Biotechnology 7:241-251 (1997); and Haseloff et al. Nature, 334:585-591 (1988).

Another method of suppression involves RNA interference (RNAi), also referred to as sense suppression, double stranded RNA suppression or posttranscriptional gene silencing. Introduction of nucleic acid configured in the sense orientation has been recently shown to be an effective means by which to block the transcription of target genes. For an example of the use of this method to modulate expression of endogenous genes (see, Assaad et al. Plant Mol. Bio. 22:1067-1085 (1993); Flavell Proc. Natl. Acad. Sci. USA 91:3490-3496 (1994); Stam et al. Annals Bot. 79:3-12 (1997); Napoli et al., The Plant Cell 2:279-289 (1990); Klink, et al., J Plant Growth Regul. 19(4):371-84 (2000); Matzke, et al., Curr Opin Genet Dev. 11(2):221-7 (2001); and U.S. Pat. Nos. 5,034,323, 5,231,020, and 5,283,184).

The suppressive effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% is preferred, though about 95% to absolute identity would be most preferred. As with antisense regulation, the effect can apply to any other proteins within a similar family of genes exhibiting homology or substantial homology.

For sense suppression, the introduced sequence, needing less than absolute identity, also need not be full length, relative to either the primary transcription product or fully processed mRNA. This may be preferred to avoid concurrent production of some plants that are overexpressers. A higher identity in a shorter than full length sequence compensates for a longer, less identical sequence. Furthermore, the introduced sequence need not have the same intron or exon pattern, and identity of non-coding segments will be equally effective. Normally, a sequence of the size ranges noted above for antisense regulation is used. In addition, the same gene regions noted for antisense regulation can be targeted using cosuppression technologies.

In a preferred embodiment, expression of a nucleic acid of interest can be suppressed by the simultaneous expression of both sense and antisense constructs (Waterhouse et al., Proc. Natl. Acad. Sci. USA 95:13959-13964 (1998). See also Tabara et al. Science 282:430-431 (1998).

Alternatively, RopGAP activity may be modulated by eliminating the proteins that are required for RopGAP cell-specific gene expression. Thus, expression of regulatory proteins and/or the sequences that control RopGAP gene expression can be modulated using the methods described here.

Another method is use of engineered tRNA suppression ofRopGAP mRNA translation. This method involves the use of suppressor tRNAs to transactivate target genes containing premature stop codons (see, Betzner et al Plant J. 11:587-595 (1997); and Choisne et al Plant J. 11:597-604 (1997). A plant line containing a constitutively expressed RopGAP gene that contains an amber stop codon is first created. Multiple lines of plants, each containing tRNA suppressor gene constructs under the direction of cell-type specific promoters are also generated. The tRNA gene construct is then crossed into the RopGAP line to activate RopGAP activity in a targeted manner. These tRNA suppressor lines could also be used to target the expression of any type of gene to the same cell or tissue types.

RopGAP proteins may form homogeneous or heterologous complexes in vivo. Thus, production of dominant-negative forms of RopGAP polypeptides that are defective in their abilities to bind to other proteins in the complex is a convenient means to inhibit endogenous RopGAP activity. This approach involves transformation of plants with constructs encoding mutant RopGAP polypeptides that form defective complexes and thereby prevent the complex from forming properly. The mutant polypeptide may vary from the naturally occurring sequence at the primary structure level by amino acid substitutions, additions, deletions, and the like. These modifications can be used in a number of combinations to produce the final modified protein chain. Use of dominant negative mutants to inactivate target genes is described in Mizukami et al. Plant Cell 8:831-845 (1996). An exemplary dominant negative RopGAP polypeptide has a non-conservative mutation of an invariant arginine residue within the GAP domain.

Another strategy to affect the ability of an RopGAP protein to interact with itself or with other proteins involves the use of antibodies specific to RopGAP. In this method cell-specific expression of RopGAP-specific antibodies is used inactivate functional domains through antibody:antigen recognition (see, Hupp et al. Cell 83:237-245 (1995)).

After plants with reduced RopGAP activity are identified, a recombinant construct comprising a RopGAP transcript under the control of a heterologous promoter can be introduced using the methods discussed below. Alternatively, native RopGAP expression can be down-regulated using an antisense construct under the control of a heterologous promoter. In these methods, the level of RopGAP activity can be regulated to produce preferred plant phenotypes.

V. Use of Nucleic Acids of the Invention to Enhance RopGAP Expression

Isolated sequences prepared as described herein can also be used to introduce expression of a particular RopGAP nucleic acid to enhance or increase endogenous gene expression. Enhanced expression can therefore be used to control plant phenotypes (i.e. senescence, submergence tolerance, defense responses, cell wall differentiation, and response to other biotic and abiotic stimuli, etc.) by controlling Rop GTPase activity under RopGAP's control in desired tissues, cells or subcellular locations. Where overexpression of a gene is desired, the desired gene from a different species may be used to decrease potential sense suppression effects.

One of skill will recognize that the polypeptides encoded by the genes of the invention, like other proteins, have different domains that perform different functions. Thus, the gene sequences need not be full length, so long as the desired functional domain of the protein is expressed.

Modified protein chains can also be readily designed utilizing various recombinant DNA techniques well known to those skilled in the art and described in detail, below. For example, the chains can vary from the naturally occurring sequence at the primary structure level by amino acid substitutions, additions, deletions, and the like. These modifications can be used in a number of combinations to produce the final modified protein chain.

VI. Preparation of Recombinant Vectors

To use isolated sequences in the above techniques, recombinant DNA vectors suitable for transformation of plant cells are prepared. Techniques for transforming a wide variety of flowering plant species are well known and described in the technical and scientific literature. See, e.g., Weising et al. Ann. Rev. Genet. 22:421-477 (1988). A DNA sequence coding for the desired polypeptide, for example a cDNA sequence encoding a full length protein, will preferably be combined with transcriptional and translational initiation regulatory sequences which will direct the transcription of the sequence from the gene in the intended tissues of the transformed plant.

For example, for overexpression, a plant promoter fragment may be employed which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill. Such genes include for example, A CT1 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al Plant Physiol. 104:1167-1176 (1994)), GPc1 from maize (GenBank No. X15596, Martinez et al. J. Mol. Biol 208:551-565 (1989)), and Gpc2 from maize (GenBank No. U45855, Manjunath et al., Plant Mol Biol. 33:97-112 (1997)).

Alternatively, the plant promoter may direct expression of the RopGAP nucleic acid in a specific tissue or may be otherwise under more precise environmental or developmental control. Examples of environmental conditions that may effect transcription by inducible promoters include anaerobic (low oxygen) conditions, elevated temperature, or the presence of light. Such promoters are referred to here as “inducible”. Promters that direct expression to a specific tissue are referred to as “tissue-specific” promoters. One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

Examples of promoters under developmental control include promoters that initiate transcription only (or primarily only) in certain tissues, such as fruit, seeds, flowers, roots, leaves, shoots, etc. Promoters that direct expression of nucleic acids in ovules, flowers or seeds are particularly useful in the present invention. As used herein a seed-specific promoter is one that directs expression in seed tissues. Such promoters may be, for example, ovule-specific (which includes promoters that direct expression in maternal tissues or the female gametophyte, such as egg cells or the central cell), embryo-specific, endosperm-specific, integument-specific, seed coat-specific, or some combination thereof. Examples include a promoter from the ovule-specific BELI gene described in Reiser et al. Cell 83:735-742 (1995) (GenBank No. U39944). Other suitable seed specific promoters are derived from the following genes: MAC1 from maize (Sheridan et al. Genetics 142:1009-1020 (1996), Cat3 from maize (GenBank No. L05934, Abler et al. Plant Mol. Biol. 22:10131-1038 (1993), the gene encoding oleosin 18 kD from maize (GenBank No. J05212, Lee et al. Plant Mol. Biol. 26:1981-1987 (1994)), vivparous-1 from Arabidopsis (Genbank No. U93215), the gene encoding oleosin from Arabidopsis (Genbank No. Z17657), Atmyc1 from Arabidopsis (Urao et al. Plant Mol Biol. 32:571-576 (1996), the 2s seed storage protein gene family from Arabidopsis (Conceicao et al. Plant 5:493-505 (1994)) the gene encoding oleosin 20 kD from Brassica napus (GenBank No. M63985), napA from Brassica napus (GenBank No. J02798, Josefsson et al. JBL 26:12196-1301 (1987), the napin gene family from Brassica napus (Sjodahl et al. Planta 197:264-271 (1995), the gene encoding the 2S storage protein from Brassica napus (Dasgupta et al. Gene 133:301-302 (1993)), the genes encoding oleosin A (Genbank No. U09118) and oleosin B (Genbank No. U09119) from soybean and the gene encoding low molecular weight sulphur rich protein from soybean (Choi et al. Mol Gen, Genet. 246:266-268 (1995)).

For expression of polynucleotides in the aerial vegetative organs of a plant, photosynthetic organ-specific promoters, such as the RBCS promoter (Khoudi, et al., Gene 197:343, 1997), can be used. Root-specific expression can be achieved under the control of a root-specific promoter, e.g., from the ANR1 gene (Zhang & Forde, Science, 279:407, 1998). Other examples include Hirel, et al., Plant Molecular Biology 20(2):207-218 (1992), which describes a root-specific glutamine synthetase gene from soybean and Keller, et al., The Plant Cell 3(10):1051-1061 (1991), which describes a root-specific control element in the GRP 1.8 gene of French bean. In some embodiments, the heterologous promoters of the invention are specifically expressed in one of the following regions of the root: cortex, stele, lateral meristem, zone of elongation, vascular, pre-vascular, or root cap.

Exemplary senescence-specific promoters include the promoter for WRKY6 factor (see, e.g., Robatzek et al., Genes Dev. 16(9):1139-49 (2002); the SAG12 promoter from Arabidopsis (see, e.g., Noh, et al., Plant Mol Biol. 41(2):181-94 (1999)).

Exemplary flood-specific promoters are: LE-ACS7, described in, e.g., Shiu et al., Proc Natl Acad Sci USA. 95(17):10334-9 (1998) and ADH promoters from diverse species, described in, e.g., Hoeren et al., Genetics, 149:479-490 (1998), Olive et al., Plant Mol Biol 2:673-684 (1990), Walker et al., Proc. Natl. Acad. Sci. USA, 84:6624-6629 (1987), and Dolferus et al., Plant Physiol 105:1075-1078 (1994).

An exemplary ROS-inducible promoter is the GST6 promoter, described in, e.g., Chen et al., Plant J. 10(6):955-66 (1996), Arabidopsis GST1, described in, e.g., Levine et al., Cell 79:583-589 (1994), maize Cat1 promoter, described in Guan et al., Plant J., 22(2):87-95 (2000), Arabidopsis PEXI promoter, described in, e.g., Lopez-Huertas et al., Embo J. 19(24):6770-6777 (2000).

An exemplary stomata-specific promoter is, e.g., the promoter of a modified potato KST1 (Plesch et al., Plant J. 28(4):455-64 (2001))

Exemplary defense-specific promoters include, e.g., the PR-1 promoters from Arabidopsis (see, e.g., Lebel, et al. Plant J. 16(2):223-33 (1998)) and tobacco (Eyal, et al., Plant J. 4(2):225-34 (1993)).

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from genes of the invention will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta.

VII. Production of Transgenic Plants

DNA constructs of the invention may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment.

Microinjection techniques are known in the art and well described in the scientific and patent literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Ballistic transformation techniques are described in, e.g., Klein et al. Nature 327:70-73 (1987).

Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature. See, for example Horsch et al. Science 233:496-498 (1984), and Fraley et al. Proc. Natl. Acad. Sci. USA 80:4803 (1983).

Transformed plant cells which are derived by any of the above transformation techniques can be cultured to regenerate a whole plant which possesses the transformed genotype and thus the desired phenotype such as increased seed mass. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

The nucleic acids of the invention can be used to confer desired traits on essentially any plant. Thus, the invention has use over a broad range of plants, including species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannesetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

Seed obtained from plants of the present invention can be analyzed according to well known procedures to identify plants with the desired trait. If antisense or other techniques are used to control RopGAP gene expression, Northern blot analysis can be used to screen for desired plants. In addition, the presence of fertilization independent reproductive development can be detected. Plants can be screened, for instance, for the ability to tolerate low oxygen conditions or for decreased senescence. These procedures will depend, part on the particular plant species being used, but will be carried out according to methods well known to those of skill.

The following Example is offered by way of illustration, not limitation.

### EXAMPLE

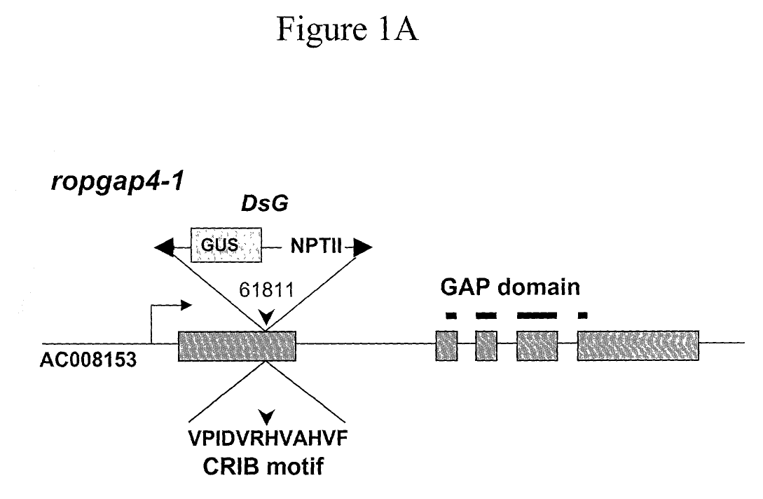

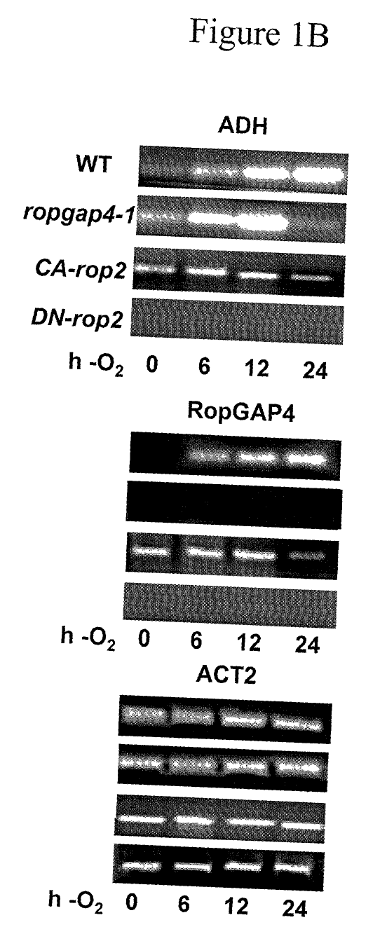

Plant endurance of transient flooding requires increased production of ATP through glycolysis and regeneration of NAD⁺ through ethanolic fermentation. Signal transduction processes that control changes in gene expression in oxygen-deprived cells involve oscillations in cytosolic free Ca²⁺. To identify genes involved in regulation of expression of the sole alcohol dehydrogenase gene (ADH) of Arabidopsis thaliana, we screened lines carrying a gene-trap transposon (DsG) (V. Sundaresan et al., Gene Develop. 9:1797-1810 (1995)) for increased GUS histochemical staining and altered induction of ADH specific activity in response to oxygen deprivation under low light. We identified a line that displayed elevated GUS staining throughout the seedling vasculature in response to low oxygen but with no apparent abnormalities under control conditions. This line contained a single DsG transposon inserted into the first exon of RopGAP4 (GTPase activating protein, 49 kDa) (FIG. 1A; GenBank AC008153; MIPS At3g11490; BAC F24K9.16, position 61811), resulting in a translational fusion within the CRIB (Cdc42/Rac-interactive binding) motif at the amino terminus of RopGAP4 (FIG. 1A). This mutant allele was designated ropgap4-1.

RopGAPs were identified in a yeast two-hybrid system based on interaction with the RHO-like small G-protein of plants, Rop (Wu, et al. Plant Physiol. 124:1625-1636 (2000)). RopGAPs possess a conserved GAP-like domain and a CRIB motif that enhances the competition between RopGAP and other Rop-interacting proteins, allowing for efficient GTP hydrolysis. Rop signaling controls intracellular Ca²⁺ gradients and actin cytoskeletal dynamics required for tip growth of pollen and polar growth of root hairs. Activation of Rop signaling is implicated in defense responses and developmental processes involving H₂O₂, whereas the inactivation of Rop signaling is necessary for abscisic acid-induced closure of leaf stomata.

RopGAP4 mRNA accumulation increased dramatically in response to O₂ deprivation in wildtype (WT) seedlings, as detected by RT-PCR (FIG. 1B). RopGAP4 mRNA was not detectable in ropgap4-1 seedlings, indicating that the DsG insertion resulted in a loss-of-function mutation.

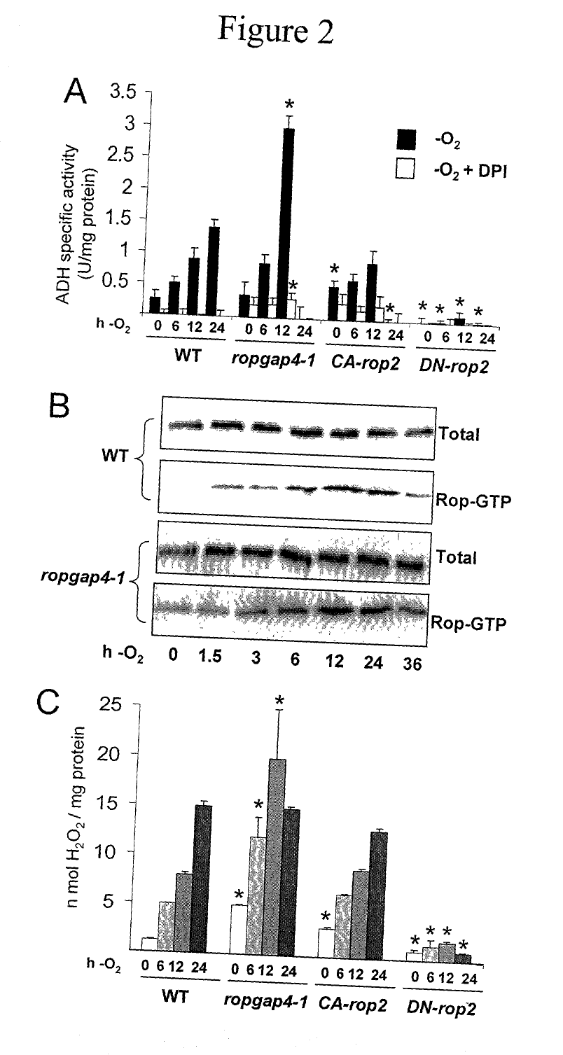

ropgap4-1 allowed us to consider whether Rop signaling is involved in regulation of ADH expression in response to oxygen deprivation. ropgap4-1 seedlings showed a more rapid and dramatic increase in ADH mRNA accumulation and 3-fold higher ADH specific activity after 12 h of oxygen deprivation than WT, but were paradoxically more sensitive to the stress (FIGS. 1C, 2A; Table 1).

TABLE 1
Effect of O₂ deprivation and DPI treatment on seedling survival +, addition
of 30 μM DPI in 3% DMSO solvent; −, addition of solvent. Data are the
mean ± SE from three independent experiments.

| | Viable Seedlings 48 h after Treatment | | | | | | | |
|---|---|---|---|---|---|---|---|---|
| Oxygen | (Percentage) | | | | | | | |
| Deprivation (h) | WT | | ropgap4-1 | | CA-rop2 | | DN-rop2 | |
| DPI | − | + | − | + | − | + | − | + |
| 0 | 100 | 100 | 100 | 100 | 100 | 100 | 100 | 90 ± 3 |
| 6 | 100 | 80 ± 5 | 90 ± 2 | 100 | 100 | 100 | 100 | 70 ± 4 |
| 12 | 100 | 0 | 0 | 50 ± 5 | 69 ± 5 | 100 | 100 | 0 |
| 24 | 80 ± 6 | 0 | 0 | 0 | 0 | 0 | 0 | 0 |

After 24 h of oxygen deprivation, ADHmRNA and specific activity levels dropped dramatically and ropgap4-1 seedlings were unable to recover upon re-oxygenation. Seedlings of a line expressing a dominant negative form of Rop2 (35S::DN-rop2 (T20N)) (Li, et al., Plant Physiol. 126:670-684(2001)) showed no detectable induction of ADH mRNA or specific activity following oxygen deprivation and increased stress sensitivity. This confirms that signaling through the Rop GTPase activates ADH expression, thereby allowing for low oxygen tolerance (Jacobs, et al., Biochem. Genet. 26:102-112 (1988)). In a line expressing a constitutive active form of Rop2 (³⁵S::CA-rop2 (G15V)) (Li, et al., Plant Physiol. 126:670-684(2001)), ADH specific activity was higher under control conditions and inducible by O₂ deprivation. The limited induction of ADH in CA-rop2 versus the excessive induction in ropgap4-1 can be explained by negative feedback regulation of Rop signaling by ROPGAP4.

The transient activation of Rop signaling by oxygen deprivation was confirmed using an assay that detects Rop-GTP by interaction with Rop-interacting CRIB motif containing protein (RIC1) (Wu, et al. Plant Cell. 13:2841-2856 (2001)). FIG. 2B compares the level of Rop in total cell extracts (Rop-GTP and Rop-GDP) to RIC 1-interacting Rop (Rop-GTP) over 36 h of oxygen deprivation. Rop-GTP levels rose in WT seedlings after 1.5 h, increased through 12 h and then decreased. Rop-GTP levels were constitutively high in ropgap4-1 seedlings and increased in response to low oxygen, but showed no decrease even after 36 h. Oxygen deprivation promotes the activation of Rop-GTP and RopGAP4 appears to negatively regulate this activation in WT seedlings.

Cotyledons of ropgap4-1 seedlings turned brown upon re-oxygenation whereas those of CA-rop2, DN-rop2 and WT remained green, leading us to suspect that ropgap4-1 seedlings succumb to oxygen deprivation and/or re-oxygenation as a result of oxidative stress. ropgap4-1 seedlings fail to control reactive oxygen species (ROS) production. We tested whether the response to oxygen deprivation was affected by treatment of seedlings with diphenylene iodonium chloride (DPI), which inhibits production of superoxide by flavin-containing NADPH oxidases and the resultant accumulation of H₂O₂. In all four genotypes, DPI reduced ADH activity under control and low oxygen conditions, demonstrating that ADH induction requires a DPI-sensitive NADPH oxidase (FIG. 2A). DPI also reduced the duration of stress that WT seedlings survived, from over 36 h to less than 12 h (Table 1). DPI treatment reduced ADH induction in ropgap4-1 seedlings and increased survival of O₂ deprivation, revealing that inability to down-regulate a DPI-sensitive NADPH oxidase reduces stress tolerance. Consistently, survival of oxygen deprivation was improved in CA-rop2 and impaired in DN-rop2 seedlings in the presence of DPI.

H₂O₂ levels increased in response to oxygen deprivation in WT, ropgap4-1 and CA-rop2 seedlings but did not change significantly in DN-rop2 seedlings (FIG. 2C), supporting a role of Rop signaling in H₂O₂ production. In WT seedlings H₂O₂ level and ADH specific activity rose coordinately over 24 h of stress. H₂O₂ levels in ropgap4-1 seedlings under control conditions and after 6 and 12 h of oxygen deprivation were significantly higher than in WT seedlings, consistent with the ADH specific activity data. High H₂O₂ in the mutant may contribute to reduced stress tolerance. In CA-rop2 seedlings, H₂O₂ levels were correlated with constitutively high ADH specific activity under control conditions but were not clearly responsible for intolerance of low oxygen.

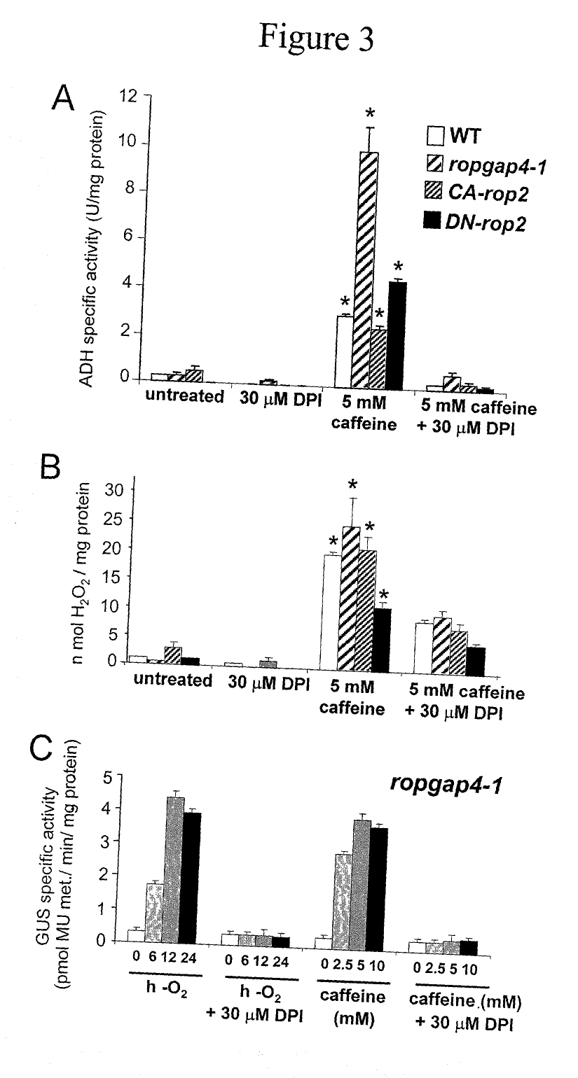

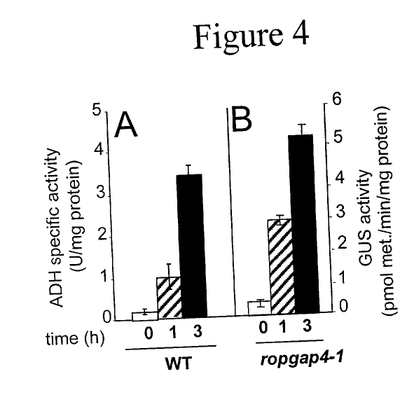

ropgap4-1 seedlings have constitutively high levels of Rop-GTP but near normal levels of ADH specific activity until deprived of oxygen, indicating that accumulation of Rop-GTP is insufficient for induction of ADH. An increase in cytosolic free Ca²⁺, due to organellar efflux and/or apoplastic influx, activates ADH expression in Arabidopsis (Sedbrook, et al. Plant Physiol. 111:243-257 (1996)). In maize, treatment of cells with low levels of caffeine stimulates ADH1 expression and promotes an increase in cytosolic free Ca²⁺, similar to that observed in response to anoxia. Caffeine treatment, under non-stress conditions, induced ADH specific activity to significantly higher levels than the maximal level observed in response to low oxygen in all four genotypes (FIG. 3A). DPI effectively blocked the caffeine-stimulated increase in ADH specific activity and the concomitant increase in H₂O₂ (FIG. 3A and B). Consistent with oxygen deprivation, the caffeine promoted increase in ADH specific activity was dramatic in ropgap4-1 and limited in CA-rop2 seedlings. In DN-rop2 seedlings, the caffeine-stimulated induction may result from a Rop-independent mechanism or interaction between a Ca²⁺ signal and the residual activity of endogenous Rops. Topical application of an H₂O₂ regenerating system, glucose and glucose oxidase, resulted in a rapid and efficient increase in ADH specific activity in WT seedlings (FIG. 4A), confirming that H₂O₂ is a second messenger in ADH regulation.

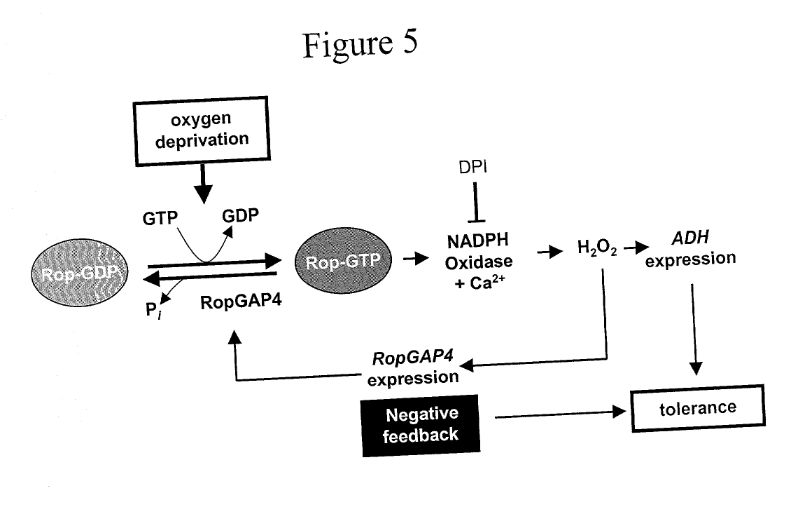

These results reveal that oxygen deprivation stimulates a Rop signal transduction pathway, activating a DPI-sensitive NADPH oxidase that results in increased H₂O₂ production, which acts as a second messenger in the induction of ADH expression (FIG. 5). An increase in cytosolic free Ca²⁺ appears to occur in this Rop-mediated signal. Without intending to limit the scope of the invention to a particular mechanism, it is believed that this could be due to the binding of Ca²⁺ by the plasma membrane DPI-sensitive NADPH oxidase gp91phox subunit (Sagi, et al. Plant Physiol. 126:1281-1290 (2001)) and/or a Ca²⁺-dependent DPI-sensitive NAD(P)H dehydrogenase/oxidase of the inner mitochondrial membrane (Moller, et al. Annu. Rev. Plant Physiol. 52:561-591 (2001)).

The attenuation of Rop signal transduction is also necessary for tolerance of oxygen deprivation. Several lines of evidence indicate that Rop signaling drives this attenuation by activation of RopGAP4 expression. First, low oxygen promoted RopGAP4 mRNA accumulation in WT but not DN-rop2 seedlings (FIG. 1B). Second, GUS activity rose in ropgap4-1 seedlings in response to low oxygen and caffeine, but was blocked by DPI (FIG. 3C). Third, application of an H₂O₂ regenerating system elevated GUS activity in ropgap4-1 seedlings (FIG. 4B). Fourth, RopGAP4 mRNA levels were constitutively elevated in CA-rop2 seedlings (FIG. 1B).

Thus, a Rop rheostat regulates the production of H₂O₂ that is required to trigger expression of beneficial genes (e.g. ADH) and avoidance of H₂O₂-induced cell death. Rop signaling is controlled by negative feedback regulation through the stimulation of RopGAP4 transcription by H₂O₂. The termination of Rop signaling by RopGAP4 would alleviate oxidative stress and limit consumption of carbohydrate reserves via glycolysis and ethanolic fermentation. The reduced oxygen deprivation tolerance of the DN-rop2, CA-rop2 and ropgap4-1 seedlings underscores the requirement for a fully functional Rop rheostat. We propose that a Rop rheostat controls developmental processes and environmental stress responses that utilize H₂O₂ as a second messenger or enhance H₂O₂ accumulation, including the response to ABA, auxin, pathogen infection and numerous abiotic stresses. The manipulation of the Rop signal transduction rheostat will enhance the productivity of crops that undergo transient submergence or soil waterlogging.

Materials and Methods

Arabidopsis thaliana ecotype Landsberg erecta (homozygous ropgap4-1, wildtype (WT)) and Columbia (homozygous CA-rop2, homozygous DN-rop2) (Li, et al. Plant Physiol. 126:670-684 (2001)) seeds were surface sterilized, incubated at 4° C. for 2 d and grown for 7 d on solid MS medium (0.43% MS salts (Sigma), 1% (w/v) sucrose, 0.3% (w/v) phytagel (Sigma), adjusted to pH 5.7 with NaOH) under 16 h illumination at 22° C. in petri dishes held vertical in racks. For oxygen deprivation, plates were transferred to an airtight chamber designed to allow entry and exit of 99.998% argon under 0.4 μmols/s¹m² per μÅ of light. For chemical treatments, 30 μM diphenylene iodonium chloride (DPI, Sigma) in 3% (v/v) dimethylsulfoxide (DMSO) or 2.5 mM glucose and 2.5 U/ml glucose oxidase in 0.9% NaCl, 10 mM Tris-HCl (pH 7.5) was applied directly to seedlings. Caffeine (5 mM) was incorporated into the medium of plates to which seedlings were transferred. Plates were returned the growth chamber for 24 h. Control seedlings were treated with 3% (v/v) DMSO or transferred to plates not containing caffeine. Seedlings were used immediately for histochemical staining or frozen in liquid nitrogen and stored at −80° C.

Histochemical staining for GUS activity was for 2 d using 5-Bromo-4-chloro-3-indoxyl-beta-D-glucronic acid. GUS specific activity was determined from cell extracts using 4-methyl umbelliferyl beta-D-glucuronide as substrate (Bailey-Serres, et al Plant Physiol. 112:685-695 (1996)). ADH specific activity was determined from cell extracts in the ethanol oxidase direction.

Arabidopsis lines with a DsG transposon were screened to identify genes that are expressed at elevated levels in response to oxygen deprivation. The DsG insertion site in ropgap4-1 (Gene Trap Riverside #27, GTR27) was determined following thermal asymmetric interlaced PCR amplification of the Ds termini and flanking sequence. The presence of a single DsG element in homozygous ropgap4-1 seedlings was confirmed by Southern blot analysis.

Total RNA was isolated by use of a RNeasy plant mini kit (Qiagen). Reverse transcription-PCR (RT-PCR) was performed with specific primers essentially as described. First strand cDNA synthesis was completed in a 50 μl reaction containing 2.5 μM oligo-dT (Promega), 5 μg of total RNA, 10 mM DTT, 1 unit/μl RNAsin (Promega), 0.20 mM dNTP mix, 5× reaction buffer (Promega), and 10 units/μl avian myeloblastosis virus reverse transcriptase (Promega), at 37° C. for 60 min, and terminated by heating to 99° C. for 5 min. PCR reactions contained 2 mM MgCl, 0.25 units Taq polymerase and 2 mM of each primer pair:

| | |
|---|---|
| ROPGAP4-forward | 5′-AGAGGGAACATCGTGCCTAC-3′, |
| ROPGAP4-reverse | 5′-AACTGTCTACTGCTGCTCTG-3′, |
| ADH-forward | 5′-TCTACTGGGTTAGGAGCAAC-3′, |
| ADH-reverse | 5′-TTGATTTCCGAGAATGGCAC-3′, |
| ACT2-forward | 5′-AAAAATGGCCGATGGT-3′, |
| ACT2-reverse | 5′-CTGGTTCGTGGTGGTGAGTTT-3′ |

PCR amplification was carried out at 94° C. for 30 sec 53.6° C. (ROPGAP4) or 55° C. (ADH and ACT2) for 30 sec and 72° C. for 30 s for 25 cycles. An equal volume of each reaction was analyzed on a 1.5% (w/v) agarose gel.

The RIC1-maltose-binding protein (MBP) fusion protein was overexpressed in E. coli and purified (K. L. Guan, et al. Anal Biochem. 192:262-267 (1991)). ROP 1-GST was used to affinity purify ROP1 antibody, which indiscriminately recognizes all Arabidopsis Rops, for immunoblotting (Sambrook et al). To assay levels of Rop-GTP, tissue (1 g) was ground under liquid nitrogen, hydrated in 2 ml of extraction buffer (25 mM Hepes (pH 7.4), 10 mM MgCl₂, 100 mM NaCl, 5 mM sodium fluoride, 1 mM sodium orthovanadate (Sigma), 1 μM PMSF, 10 μg/ml aprotinin (Sigma), 10 μg/ml leupeptin (Sigma), 1% (v/v) Triton X-100) and centrifuged at 13,000×g for 10 min at 4° C. Thirty μl of supernatant was conserved (Total Rop) and the remainder mixed with 2 ml of extraction buffer lacking Triton X-100 and 30 μl of MBP-RIC1 beads, gently shaken for 3 h at 4° C. and centrifuged at 500×g for 5 min. The pellet was washed three times with 3 ml buffer (25 mM Hepes (pH 7.4), 1 mM EDTA, 5 mM MgCl₂, 1 mM DTT, 0.5% (v/v) Triton X-100). Proteins were fractionated by 15% SDS-PAGE, transferred to nitrocellulose and immunoblots incubated with affinity purified anti-ROP1 antibody (1:300), followed by goat anti-rabbit IgG horseradish peroxidase-conjugate (1:7,500) with chemiluminescence detection using ECL reagent (Amersham Pharmacia Biotech Inc) and x-ray film (Hyperfilm, Amersham).

H₂O₂ levels were measured using a modification of the Terashima (1998) method (Terashima et al., Physiol. Plant. 103:295-303 (1998)). Tissue (0.5 g) was homogenized under liquid nitrogen, hydrated in 3 ml of 5% (w/v) metaphosphoric acid and centrifuged at 1,500×g at 4° C. for 20 min. The pH of the supernatant was adjusted to pH 7 with 1M Tricine, 6M NaOH. H₂O₂ levels were measured in a 500-μl reaction containing 200 mM MOPS-KOH (pH 7.8), 50 mM NADH in 10 mM potassium phosphate (pH 7.8). Absorbance at 340 nm was measured, 0.5 U of Streptococcusfaecalis NADH peroxidase (Sigma) was added, the sample was incubated for 1 h and absorbance at 340 nm was re-measured. H₂O₂ levels were determined from the decrease in absorbance in 60 min using a standard curve generated from similarly processed H₂O₂ standards.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A nucleic acid comprising a heterologous plant promoter operably
linked to a polynucleotide encoding a Rop GAP polypeptide, wherein
the RopGAP polypeptide comprises a Cdc42/Rac-interactive binding (CRIB) motif and a GAP domain;
the RopGAP polypeptide inactivates Rop GTPase signaling; and
the heterologous promoter is expressed in a plant tissue other than pollen.

2. The nucleic acid of claim 1, wherein the RopGAP polypeptide is selected from the group consisting of Arabidopsis RopGAPl (SEQ ID NO:2), Arabidopsis RopGAP2 (SEQ ID NO:4), Arabidopsis RopGAP3 (SEQ ID NO:6), Arabidopsis RopGAP4 (SEQ ID NO:8), Arabidopsis RopGAP5 (SEQ ID NO:10) and Arabidopsis RopGAP6 (SEQ ID NO:12).

3. The nucleic acid of claim 1, wherein the heterologous promoter comprises a ROS-inducible element.

4. The nucleic acid of claim 1, wherein the heterologous promoter induces expression of the polynucleotide in the presence of less ROS than required to induce a native RopGAP promoter.

5. The nucleic acid of claim 4, wherein the heterologous promoter comprises an ADH promoter.

6. The nucleic acid of claim 1, wherein the heterologous promoter induces expression of the polynucleotide in the presence of more ROS than required to induce a native RopGAP promoter.

7. The nucleic acid of claim 1, wherein the polynucleotide is in a sense orientation compared to the heterologous promoter.

8. The nucleic acid of claim 1, wherein the polynucleotide is in an antisense orientation compared to the heterologous promoter.

9. The nucleic acid of claim 1, wherein the heterologous promoter is seed-specific.

10. The nucleic acid of claim 1, wherein the heterologous promoter is endosperm-specific.

11. The nucleic acid of claim 1, wherein the heterologous promoter is embryo-specific.

12. The nucleic acid of claim 1, wherein the heterologous promoter is root-specific.

13. The nucleic acid of claim 1, wherein the heterologous promoter is a senescence-specific promoter.

14. A nucleic acid comprising a heterologous plant promoter operably linked to a polynucleotide encoding a dominant negative RopGAP polypeptide.

15. The nucleic acid of claim 14, wherein the dominant negative RopGAP polypeptide comprises an amino acid sequences at least 80% identical to SEQ ID NO: 13 and SEQ ID NO:14.

16. The nucleic acid of claim 14, wherein the dominant negative RopGAP polypeptide is a RopGAP polypeptide lacking a GAP domain.

17. The nucleic acid of claim 14, wherein the dominant negative RopGAP polypeptide a conserved arginine residue of a RopGAP GAP domain is altered.

18. A plant comprising a heterologous promoter operably linked to a polynucleotide encoding a RopGAP polypeptide, wherein
the RopGAP polypeptide comprises a Cdc42/Rac-interactive binding (CRIB) motif and a GAP domain;
the RopGAP polypeptide inactivates Rop GTPase signaling; and
the heterologous promoter is expressed in a plant tissue other than pollen.

19. The plant of claim 18, wherein the RopGAP polypeptide is selected from the group consisting of Arabidopsis RopGAP1 (SEQ ID NO:2), Arabidopsis RopGAP2 (SEQ ID NO:4), Arabidopsis RopGAP3 (SEQ ID NO:6), Arabidopsis RopGAP4 (SEQ ID NO:8), Arabidopsis RopGAP5 (SEQ ID NO:10) and Arabidopsis RopGAP6 (SEQ ID NO:12).

20. The plant of claim 18, wherein the heterologous promoter comprises a ROS-inducible element.

21. The plant of claim 18, wherein the heterologous promoter induces expression of the polynucleotide in the presence of less ROS than required to induce a native RopGAP promoter.

22. The plant of claim 21, wherein the heterologous promoter comprises an ADH promoter.

23. The plant of claim 18, wherein the plant has increased tolerance for low oxygen levels than a nontransgenic plant.

24. The plant of claim 18, wherein the heterologous promoter induces expression of the polynucleotide in the presence of more ROS than required to induce a native RopGAP promoter.

25. The plant of claim 18, wherein the polynucleotide is in a sense orientation compared to the heterologous promoter.

26. The plant of claim 18, wherein the polynucleotide is in an antisense orientation compared to the heterologous promoter.

27. The plant of claim 18, wherein the plant is a rice plant.

28. The plant of claim 18, wherein the heterologous promoter is seed-specific.

29. The plant of claim 18, wherein the heterologous promoter is root-specific.

30. The plant of claim 18, wherein the heterologous promoter is senescence-specific.

31. The plant of claim 18, wherein the plant has increased tolerance for reactive oxygen species levels than a nontransgenic plant.

32. The plant of claim 18, wherein the plant has increased tolerance for biotic or abiotic stresses delayed scenescence than a nontransgenic plant.

33. The plant of claim 18, wherein the plant has delayed scenescence compared to a nontransgenic plant.

34. A plant comprising a heterologous plant promoter operably linked to a polynucleotide encoding a dominant negative RopGAP polypeptide.

35. The plant of claim 34, wherein the dominant negative RopGAP polypeptide comprises an amino acid sequences at least 80% identical to SEQ ID NO: 13 and SEQ ID NO:14.

36. The plant of claim 34, wherein the dominant negative RopGAP polypeptide is a RopGAP polypeptide lacking a GAP domain.

37. The plant of claim 34, wherein the dominant negative RopGAP polypeptide a conserved arginine residue of a RopGAP GAP domain is altered.

38. A method of modulating negative feedback regulation of a Rop GTPase in a plant, the method comprising,
introducing an expression cassette comprising a heterologous promoter operably linked to a polynucleotide encoding a RopGAP polypeptide, wherein
the RopGAP polypeptide comprises a Cdc42/Rac-interactive binding (CRIB) motif and a GAP domain;
the RopGAP polypeptide inactivates Rop GTPase signaling; and
the heterologous promoter is expressed in a plant tissue other than pollen.

39. The method of claim 38, wherein the RopGAP polypeptide is selected from the group consisting of Arabidopsis RopGAP1 (SEQ ID NO:2), Arabidopsis RopGAP2 (SEQ ID NO:4), Arabidopsis RopGAP3 (SEQ ID NO:6), Arabidopsis RopGAP4 (SEQ ID NO:8), Arabidopsis RopGAP5 (SEQ ID NO: 10) and Arabidopsis RopGAP6 (SEQ ID NO: 12).

40. The method of claim 38, wherein the heterologous promoter comprises a ROS-inducible element.

41. The method of claim 38, wherein the heterologous promoter induces expression of the polynucleotide in the presence of less ROS than required to induce a native RopGAP promoter.

42. The method of claim 41, wherein the heterologous promoter comprises an ADH promoter.

43. The method of claim 41, further comprising the step of selecting a plant that has increased tolerance for low oxygen levels compared to a nontransgenic plant.

44. The method of claim 41, wherein the heterologous promoter induces expression of the polynucleotide in the presence of more ROS than required to induce a native RopGAP promoter.

45. The method of claim 41, wherein the plant is a rice plant.

46. The method of claim 41, wherein the heterologous promoter is seed-specific.

47. The method of claim 41, wherein the heterologous promoter is root-specific.

48. The method of claim 41, wherein the heterologous promoter is senescence-specific.

49. The method of claim 38, wherein the polynucleotide is in a sense orientation compared to the heterologous promoter.

50. The method of claim 38, wherein the polynucleotide is in an antisense orientation compared to the heterologous promoter.

51. The method of claim 41, further comprising the step of selecting a plant that has increased tolerance for reactive oxygen species levels compared to a nontransgenic plant.

52. A method of modulating negative feedback regulation of a Rop GTPase in a plant, the method comprising, introducing into the plant an expression cassette comprising a heterologous plant promoter operably linked to a polynucleotide encoding a dominant negative RopGAP polypeptide.

53. The method of claim 52, wherein the dominant negative RopGAP polypeptide comprises an amino acid sequences at least 80% identical to SEQ ID NO:13 and SEQ ID NO:14.

54. The method of claim 52, wherein the dominant negative RopGAP polypeptide is a RopGAP polypeptide lacking a GAP domain.

55. The method of claim 52, wherein the dominant negative RopGAP polypeptide a conserved arginine residue of a RopGAP GAP domain is altered.


SEQUENCE LISTING (24 sequences)

SEQ ID NO: 1 - DNA, 3286 bp, Arabidopsis thaliana
  RHO-like small G-protein of plants (Rop) GTPase activating protein (GAP) 1 (RopGAP1)
tttagaaaacgtgcaaaaatcataaagtatactgattttcccccaaacgaaatccacatttataagtgataaggacttgtaaacattaatatatcaatgtttatccttttatatcaatacttaatgttaactaatttaattgtaaaacctccaatagtttaactaaaatcaatcataaatatgtaacaatcagtagtattctcttttttttttgtgttaccaaaacttatcttattcaactatttttatatggtttaaccactttttatggtcaatgtttagtcatttatttaaatttaattagtagtaacaaaactctgtttttttgttcattagtggcaaccgaattcatctaattagggttttgaatcaacaatctaatcttcgatcttgagctctatttccgggtttatacatttccttcaattcaaaaccttttctttcattgacaaatgatttgaaccaaaaaagttgtttcctttgccaaaatctgaagcgccatgactgaagttcttcactttccttcatctccaagcgcttctcattcatcttcttcttcttcttcttctccttcaccttcttctttatcttacgcctctcgctctaatgcgactctcttgattagctctgaccacaaccggagaaacccagttgctagattcgatcaagatgttgactttcatgcctcaatcgaagaacaagatttgagaagacggagcagtaccgatggaggagaagaagacgatggtggggaagatcagatttcgttgttggctcttcttgttgccattttcaggagatctttgatttcttgcaagagtaaccggagggagctttgtagcatggagattggatggcctaccaatgtcagacacgtggcgcacgttacctttgatcgtttcaatggcttcttgggtttgcctgttgaattcgagcctgaagttcctagaagagctccaagcgccaggtacattgtccaaagatcctacctttttattgaattgctcttaaaagtctcctcacaacaatgatcttgtcctgcaattttggggtgggtgagcagatggatatagaatgcttctgattaaggcttccttgtttcgagttgtagaacctctaagttttggttgtatacagattaaggtttatgtggaattagtgaaaccaaaacgagaagaacttatttgatttttgattcctttatcagtcagtacatgaagtgattagagcacgttcttgtctctctctgttttttcagtctcatagctatgtgtttccgtggaagctaaacacttcatatttacaagattgggctattgagccatgaaccaatgtgttcattcttgcagttatactatagttacttgtcttcaaacatctatatttgatgtcttagatactatctcttagttgttagattctgtgaaggttgcattgttcatccaactatttacatgcttaatcgataagatcaatgttgaaaagggtgaaactgcgcctatgcaattgtaactttgctcttctcggaatatttgttttggaagtataaattgtctctcgtgtgctatcccatctcttacctcttttggtatctcattactttagtgcaacagtctttggggtatcaaccgaatcaatgcaattatcgtatgattcaagaggcaattgtgtaccaaccatactattgctgatgcaaaactgtttatatagtcaaggaggcttgcaggtaatgtacattgcttcatcgagtttactccaaagtgaatatattataagctttccatgtaatcatttcttcatttatttttttaggcagagggcatttttagactcactgctgagaatagtgaggaagaggcggttagggaacaattaaaccgaggatttatacctgagcgaatcgatgttcactgtttggcagggcttatcaaggtatgtaacatatgcacatcttatttccaaaacttatatatgaaccttggtttttaattctggttttcggtttcaggcatggtttagagaactgccgacaagcgttcttgattcgttgtcgcctgaacaggtgatgcagtgccaaacagaagaggaaaatgttgagctcgttaggcttcttccacctacagaagctgctctacttgattgggccatcaatctaatggcagatgttgttcagtatgaacatctaaacaagatgaattcacgcaacatcgctatggttttcgcaccaaatatgacacaggtgagtctgcaacaacaaggttgattatcaatgtttaactcggggtcagatgggataaaatgattgagaaacacttgtattgtagatggatgatccactgacagcactgatgtatgcggttcaagtgatgaactttctcaagacactaatcgaaaaaactttaagagaaaggcaagactcagtggtcgagcaagctcatgcattccctttagaaccgtctgatgagagtggtcaccaaagcccttcacaatctttggcttttaacaccagtgagcagagtgaagagacgcaatcagacaacatcgaaaatgctgaaaatcagagttcaagcagtgagatatcagacgaattaaccctagagaacaatgcatgtgaacagagagaaacagactttggaaaatacagaacaggaagattgagcgactcgagtcaacaggtggtgctgaatctagatcctccagctcagtggccagtgggcagaacaaaggggttgaccaacttgagccgtgtaggatcgagggtagagcgtactgaagcttggcggtgaagaaaaaggagcagagagctccttgaggagacttaagctgttcttcgaaatcaaatgttgtttgattcgttttgtatgtgttaatgtggttctgttggtgtcattgttctatattttttggtcaatgttctaaattggcttcttctgtgttaaaaccgtccataccgattcttaaccaaacttgtaaaccgtattgaaccagtgtctctttcgggcttctcacgatgaaatggtgaagcatgttcttcctgtctacctttggtattggtcgtgaaagctacaaatttgcatatttggaaccaaataaagatgatatattaatattgttatgtaactattttttacttgttttgatactagtcacaaaattgaaatattgatatgaatagtgtacaaaataaaagttgagataatggcaattgagaaagatggatttaaaattggttcttcaatggaccaagggacaaaagtgcatgacaaaagcatattgtagaggagaa

SEQ ID NO: 2 - protein, 466 aa, Arabidopsis thaliana
  RHO-like small G-protein of plants (Rop) GTPase activating protein (GAP) 1 (RopGAP1)
MTEVLHFPSSPSASHSSSSSSSSPSPSSLSYASRSNATLLISSDHNRRNPVARFDQDVDFHASIEEQDLRRRSSTDGGEEDDGGEDQISLLALLVAIFRRSLISCKSNRRELCSMEIGWPTNVRHVAHVTFDRFNGFLGLPVEFEPEVPRRAPSASATVFGVSTESMQLSYDSRGNCVPTILLLMQNCLYSQGGLQAEGIFRLTAENSEEEAVREQLNRGFIPERIDVHCLAGLIKAWFRELPTSVLDSLSPEQVMQCQTEEENVELVRLLPPTEAALLDWAINLMADVVQYEHLNKMNSRNIAMVFAPNMTQMDDPLTALMYAVQVMNFLKTLIEKTLRERQDSVVEQAHAFPLEPSDESGHQSPSQSLAFNTSEQSEETQSDNIENAENQSSSSEISDELTLENNACEQRETDFGKYRTGRLSDSSQQVVLNLDPPAQWPVGRTKGLTNLSRVGSRVERTEAWR

SEQ ID NO: 3 - DNA, 2535 bp, Arabidopsis thaliana
  RHO-like small G-protein of plants (Rop) GTPase activating protein (GAP) 2 (RopGAP2)
tcgaagattttaaagagggaaaagagagagaaaacatggattcaatattaatagtgaaaatatttatatatccaaaataaaatgtcaatttgacaaagaaaaaaatcaaaaaatcaagtagatccataaatgaaacgccacgtcaacaaccaacaaccaattaaattgagtggcattatcgtagatttagagaagttctttggggtaaaacgtgattccaataaaagctacattagataaataatatccctagagggagaaaattttgaaatgagtggaccgttgctttgaaatccaaccaaaaagacaaaaatttcaaaaccaaacatatattatattaaatcacataaaagaataaaaaagaaagagaaaccttttcgagaatttcaaaaatcaaattcacttccatagagagagaagaagaagcacaaagtccagagagaaagagagagagaagctgcgaaaatctttcatagttctcaatgacggggctagtgatgatgactaaaggcggaggttgcggcggcggaggaaaaggaggaaggagaaaatcaacggcggaggaggaagaagaagaagagcagaatcagcaacaactgtctcttgttgagtttctcttaacggcgctgcgtaaatctgtggtttcttgccgtgtagacaacagacaagacgacggcggagtaggcggagggatttcgtccgccgttcatcacatggagatcggttggccaacaaatgttcgacacatcactcatgtaacattcgatcgattccatggctttcttggtctccctcacgagcttcaagttgagatcccatgtcgagtccctagtgcaaggtaatctctcaaaatttcactactgctttagaatctgaaactggatctgaaattgtagcttcttatttaatggtggattctctttgattaatgcagtgtgagtgtgtttggtgtatcggcggaatcgatgcaatgttcttatgatgagaaaggaaacagtgtcccaacgattctattacttatgcaggagagactctattctcaacaaggtcttaaggtaatgggaattggtaatcaattttgttgagaaatttgttgagaggagactgaatttgatatcttgcttttgttgtgtaggctgaaggaattttcaggataaaccctgagaatagtcaagaggaacatgtaagagaccaactaaacagaggtattgtacctgagaatattgatgtgcattgtttggctggtcttattaaagcttggtttagagagttaccaagtggagtgcttgatggtctttctcctgaggaagttctcaattgcaacactgaggatgaatccgttgaactcattaagcagttgaagccaactgagtctgctttgcttaattgggctgttgatcttatggctgatgttgttgaagaagaagagtctaacaaaatgaatgcaaggaatatagccatggtttttgctcctaatatgactcaggtaaaaacaaagttaacctttgttcaatgaataagaatcactgagtagttactaacatttgattcttgcattctctaactgtagatgacagatccattaacggctcttatgcatgctgttcaagtgatgaacttgcttaagactcttatcacaaagacactagctgaacgggaagaaaatgctaccggatcagaaggatattctccatcccactcatccaattctcaaactgattctgattctgacaatgcacaagacatggaagtcagctgtgaatcacaagccacagattcggaatgcggagaagaagaagaagtagaggaagtagaacagcatcaagaacatctcagccgccactctacccacgaagatgaaaccgatattggatcattatgttcaatagagaaatgcttcttgaatcaactcaacaacaatgctgctagagtttcaaacaccagtatctctgaagactggagtcctaaagcatttccgcttgtatcattcacagaaaacaaaagcaacactttgagctcaagcactagcgactaagattcaagcctggtctgcatttcccacgctttcaccagtgacaacctcatttctatgtagtttctgttcaatctgctgcagagaagaaacaagaaggcagaaaaacagctttgtgtctgtaaatcaatacagaaattattgtccaattcgttagggtctttagggattgttggtgagaagtctgattttagggttcaacaagggctgaacctgtctgtgttgactgttcttgttgttgttgtcgtccctctccatgtttgtgtgtctgtctgtttctgtttgttgtgatgtttggttttgtagcaaagataatctgatatttctctgttatttttgaacaacactgaatctttcttctaaactctaaagacaacaacagatcattaaaaaatgatagaagaagaattcaatgaatttaatgttaataaatcataaaatttctacatgagtttcaataataaacataaattacaaattgttatttgcagattaaaaaaaaa

SEQ ID NO: 4 - protein, 424 aa, Arabidopsis thaliana
  RHO-like small G-protein of plants (Rop) GTPase activating protein (GAP) 2 (RopGAP2)
MTKGGGCGGGGKGGRRKSTAEEEEEEEQNQQQLSLVEFLLTALRKSVVSCRVDNRQDDGGVGGGISSAVHHMEIGWPTNVRHITHVTFDRFHGFLGLPHELQVEIPCRVPSASVSVFGVSAESMQCSYDEKGNSVPTILLLMQERLYSQQGLKAEGIFRINPENSQEEHVRDQLNRGIVPENIDVHCLAGLIKAWFRELPSGVLDGLSPEEVLNCNTEDESVELIKQLKPTESALLNWAVDLMADVVEEEESNKMNARNIAMVFAPNMTQMTDPLTALMHAVQVMNLLKTLITKTLAEREENATGSEGYSPSHSSNSQTDSDSDNAQDMEVSCESQATDSECGEEEEVEEVEQHQEHLSRHSTHEDETDIGSLCSIEKCFLNQLNNNAARVSNTSISEDWSPKAFPLVSFTENKSNTLSSSTSD

SEQ ID NO: 5 - DNA, 3052 bp, Arabidopsis thaliana
  RHO-like small G-protein of plants (Rop) GTPase activating protein (GAP) 3 (RopGAP3)
atgactaacttctcccgatccaaatccaccggaactatcgggttcccggagtttaaacccactcgacccggtccagacaaatacgagaacatccacaacgatgacgacgagtacgaagaaggacatagcactacgagtacagactactacgacgcttcaacgccgttgagctcacacgcgtcaagatccgggaacgggtccggctcgggtcagttaacggtcgtagaccttcttgcggcggttctgaggaagtcgttggtgatgtcgtgtgcgatggagagaggggaagatgacgtggtggcgtctatggatataggttggccgacggaggttaagcacgtgagtcacgtgacttttgatcggttcaatggttttcttggtcttccttctgagctcgaaccggaggttcctcctcgagctcctagcgccaggtccgttattaattaattattatcccaaagatgagtaatattccaaacaaaaaatgaatttaatattttattccatatatattgaaaaaagattatgatatgaggaattaaataaatattttcctagctggaaatctgtatatggcatgttactgtgttgggtagaatcattaagatggttgttgtttggatttaaatgcatgattacgacactcgtttcttcacaaaagaagattctctttttgtttttttttctctcttcaaagattttcaatggtatgtaaagtcaataaccatttattcaaacaatgaaactatttagacataaatttggtctttaatcaactcatgaagtagtggttgaatgacacatcctggaattggggattagtgtttaagctttcacaacataatgatattatttttgggctttccctagagattggtttgtatgatgatacatggagctctaataagtttggtttgtatagaagatacaaacttttgggtcctatggagatatactaacagaaacacgattacacaacaaatttctcataaaaaaaaaagtaaacaatttgtaaaatcgaatttttcccctaatatgtacggctaaaagaatggaaatcatgaatttttcaaaaagatataattggtcttcttgaattgtgaaacttatggctttccgtacgtagatggatttgtgaagtaaaagagtgactgattatttttgatgatacttgtgagtttcactaagttggtgactttgtatatgatataatgattggttctttctaacgattgagcttatatgttttctctagatttatttctataagagtaaaatataggtttttatatgatctaacatattggtttttctatgagtaaagatttcttattttaattcagtgtaagtgtatttggagtatcggcgaagtcaatgcaatgctcttacgacgacagaggaaatagtgtcccaacaatccttctcaggatgcaaaaacgtctatacactgaaggaggtcttaaagtaagtctctaccctcttctgttccgtgaaaatttcggattagtttggaaaagagtttgatataatgtgtgattgtttatttaccttaaggcagaaggaattttccgaataaatccagacaacggcaaagaagagcatgtccgtagacagttgaactgtggtgtggtaccacgtggaattgatgttcattgcttggcgggtttaataaaggttgaaaaaggaaagatttaaattagtttctataacattttaagttcaaaaggtttgatgtttgctgttgttgaatttcggattaaattaggcgtggttcagggagctacctacaggagtgcttgatgtgttgacaccagagcaagtaatgaggtgtaacacggaggaggattgtagccggctagtgatacttcttcctccggttgaatctgcgattcttgattgggccatcggtctaatggcggatgtggtggagcatgaacagtttaacaaaatgaatgctcgcaatgtagctatggtctttgctccaaatatgactcaagtcagtaataaaattgaattttcttttaatgaaacagttgattctttagttatatagttttgaatctcatgaaaatgttttgatgttgtagatggcagatcctttaactgcactcatccacgcagtgcaagtgatgaactttctcaagactttgatcttaatgaacctcaaagaaagggaaaacgcagacgcgaaagcaaggtggctcaagaaacaaacatctgatccatcagaagaatgggaatcacaacactccgagatattaagccctgagaaaccaaacaataataaccctaagttcttgagagtggctactctgtgtaggctagaggctgacaatgaagaagagttttggaatataaagaagagaaacgatcatgaaggtgttttagatacttcatcgggtaatggaaatattggaccggtacaaaggttgtgtaagcatccattgtttcagttaagcaaatccaccaagaaagcttttgtaagtaatcgagatgaaggaagaaaaggacgagaagcttggagttcacgtctctcttctttgccttggtaaaattttcttacctcactttatttttcatgtttttgagagtgtaaaaaatattgtgaaatatgattataggtgttagttgtttgaaaatagggataaatttacatccaatcaattcagaaatggaaacaaatgtatcctagttactttctgagagaaaactcatactctattcacaaaaacagagcattccaacaacagtcataacagagttgtcgtctgtttctattagcgaattttaaagaattgatatcgtatgtagaatatcaggcgatatttagaaaaatcagatcaaagaaggttatatgagcatatgaccactagaccggctagtatcaaaatattccaggtaattaagtctcaaaatcagatatagatcttgtaagctgtgttagatatttaatttgagattcttgaaacaaataccaaaaaataacaaaattcaaatctcaataattgttggaagaatgttcccgtacatccgaattaaatcacgcattcaa

SEQ ID NO: 6 - protein, 455 aa, Arabidopsis thaliana
  RHO-like small G-protein of plants (Rop) GTPase activating protein (GAP) 3 (RopGAP3)
MTNFSRSKSTGTIGFPEFKPTRPGPDKYENIHNDDDEYEEGHSTTSTDYYDASTPLSSHASRSGNGSGSGQLTVVDLLAAVLRKSLVMSCAMERGEDDVVASMDIGWPTEVKHVSHVTFDRFNGFLGLPSELEPEVPPRAPSASVSVFGVSAKSMQCSYDDRGNSVPTILLRMQKRLYTEGGLKAEGIFRINPDNGKEEHVRRQLNCGVVPRGIDVHCLAGLIKAWFRELPTGVLDVLTPEQVMRCNTEEDCSRLVILLPPVESAILDWAIGLMADVVEHEQFNKMNARNVAMVFAPNMTQMADPLTALIHAVQVMNFLKTLILMNLKERENADAKARWLKKQTSDPSEEWESQHSEILSPEKPNNNNPKFLRVATLCRLEADNEEEFWNIKKRNDHEGVLDTSSGNGNIGPVQRLCKHPLFQLSKSTKKAFVSNRDEGRKGREAWSSRLSSLPW

SEQ ID NO: 7 - DNA, 3045 bp, Arabidopsis thaliana
  RHO-like small G-protein of plants (Rop) GTPase activating protein (GAP) 4 (RopGAP4)
atagaaacaaaaagttattacataatacaaaatttgcaaggtttctttacttcataaagagaaagacaccaaaaaaaaagtaagtcacaaaataaaaaagacagctcgacattcatcttcctcgttaatccttcatcttctctcaccaacctccaccaatctctctctctttcttctctgtgtataggatacgaagaagaagaagaaagaaagaaagattgtgaagtgataattacaagccaccaactaatatcctttcttcaattcaactctacgcgtcgcttagtaatacccaaaaagttttaatcttttgagtttcccaattattttccccaacagaaatatcattaattactccactaatccaattttctaggttttctctttttccccctaaagattgattctttatagccattgattttgacctctccatcactgttttcagagagtacccatcactccaagatttgatttttttggattatcctttgagaataatggctaaagttctaaaatcgagccaatcatgtcatttcccttcaccttcaagctcttcttctacatcatgtggaggaggcaatgatggtagtaatagagacccacattctccttttaacatttctcgtcgtgaggaagaggaagaagaggaagagagaagcgagaaagagagagaaagattcgagctttcatctgcattggagattcttgtttctgctattagaaggtctgtgattggtgggtgtgttggtgaagaggatctttgttcaatggagattggtgttcctacagatgttagacatgtcgctcatgttacctttgatcgtttccatggcttccttggcttgcctgttgagtttgaacctgaagtccctagacgagctcctagtgcaaggttcgtttacttgtaacgatgatagatgtttctggtaatgtttggtgattaggattctcttagttcttgttttagttaacaaattcttgggaataatgttgttgttgaagctatgtgttaaaaatcaattttgggtgtggttttggtttcttctatttccagtagttgccgtaattgaaatccccttctgttaatagaatctattctgtcttttacttgcaattagtgtgtcccctgacatcaatgatccatactttttgatgttcttgattcaaatccctttgttttctgtgagttgttaaggatcttgagtgaggtatatagattgaggacttcttttgtgaattctctatgaatttgggtgtaattattaagtgaatagctagtcaacgactcttgactgatcagattagatagtttaactcttttgtgtaaatcagaagctgattagtctttttctgtaaatgcagtgcaaccgtgtttggagtctctactgaatcaatgcagctatcttatgatactagagggaacatcgtgcctacgatacttttgatgatgcagagtcacttgtacagtagaggcggattgcgggtacgtgttaatctgcatgtttaaaacaatttccatctggtttaatgacaatgggtgaataaagttttcatttgctttgtttgttgccttatataggtagagggaattttcaggattaatggtgagaatggtcaagaagagtacataagagaagagttgaataaaggtattatacctgataacattgatgtccactgcttagcaagtctcattaaggtaaaaacccataaactccacaaggatcttattgtttcctttcccttctttcaccctttttgaatttgtggattttttcttggtttatcctctaggcttggtttagggaacttcctagtggcgttttggattcactttcaccagagcaagtaatggagtctgagagtgaagatgagtgtgtggagcttgtaaggcttcttccttcaacagaagcttctttgttagattgggctatcaatctaatggctgatgttgttgagatggaacaacttaacaagatgaatgctcgaaacattgcaatggtttttgcacctaacatgactcaggtaagttttttgaatgaatctctaaagattgtttgatgtgttcgtattttggtaaatgacatcagtcaaattgcagatgttggatccattgacggctctaatgtatgctgtgcaagttatgaactttctgaaaacacttattgtgaagacgcttaaagaccgaaaagaatctagagataagttggttccagcctcaaatccaagtcctcgggaccataatggtgatcagagcagcagtagacagttgttacatctcatgaaagctaacaaggaggaaactttagacaactttgaggcggagatgaaagacaaagaagagtctgcagatgaagaagaagaagaatgtgccgaatctgtagaacttgttgacataaaaaagtcttctctggttaataacagcagtggaggttttggacagaaacacattggttgggaggagcagagaaccatgtcgaaagcgagtagtattgtgggacgtgtgaattacagagttgagctattcgaagcttggcgatgaaaaaaggttactgttgatgtttttgatccagcttttggatttcttaatttgggttttgtgggatggatggatgcttttgaattttgatgatggttgcttgtgtatcaagaaatgctacgtttctgcatataatgtcaatgataagtttctcagtttctctttctcttcacaaggatttatcttcatacttgaaagcagtatctgcaacttttgcaggaaataaatcatttaccaaatttaccataatatattacagtcacacttttttccaatactagctccacatcatcacattgaatggcaggtgttgaaatttattcaaggaaacaaaataacagtagtaaataaatacaaatagagcaacatgacagaagttgattacttcttacttcttagtcaaggaaacgacaagactgacagtcgttttgttcctgcagttggcatgttgagtcacatggtatgcatacttatcgaacaaggtatcgataatttcttctcca

SEQ ID NO: 8 - protein, 435 aa, Arabidopsis thaliana
  RHO-like small G-protein of plants (Rop) GTPase activating protein (GAP) 4 (RopGAP4)
MAKVLKSSQSCHFPSPSSSSSTSCGGGNDGSNRDPHSPFNISRREEEEEEEERSEKERERFELSSALEILVSAIRRSVIGGCVGEEDLCSMEIGVPTDVRHVAHVTFDRFHGFLGLPVEFEPEVPRRAPSASATVFGVSTESMQLSYDTRGNIVPTILLMMQSHLYSRGGLRVEGIFRINGENGQEEYIREELNKGIIPDNIDVHCLASLIKAWFRELPSGVLDSLSPEQVMESESEDECVELVRLLPSTEASLLDWAINLMADVVEMEQLNKMNARNIAMVFAPNMTQMLDPLTALMYAVQVMNFLKTLIVKTLKDRKESRDKLVPASNPSPRDHNGDQSSSRQLLHLMKANKEETLDNFEAEMKDKEESADEEEEECAESVELVDIKKSSLVNNSSGGFGQKHIGWEEQRTMSKASSIVGRVNYRVELFEAWR

SEQ ID NO: 9 - DNA, 3131 bp, Arabidopsis thaliana
  RHO-like small G-protein of plants (Rop) GTPase activating protein (GAP) 5 (RopGAP5)
gtcataatgaccccctccgaattgagaatttatttttcacggctctttagatgctttttttgggttcggtagaagtatataactcacggttaagcttttatagaatctcaatattaatttgtccatcaaggcatcaaaataatgtttagattaccatctcagcccaataccggccataatcttatatggaggatgcatattattatgtagacatcaatgatcaatcactttgggacaactactcaagctcgttttctaacgaattgggatttttgaattacttactcaaaatagtgtagtcggttttggtctagcatgatctaaaaagtccaacaagccgagaggcccaaaaccctaatattcaaaacttcgtggcctaaatgtttcatcctgtgtaattttcttcaacaaccattatcttgggtgaaaattgtcaaatttgcatattttccattattgtcattgactttctgattcggtaacagtcatatgatgtaaaaatgggccacatccttatttggtttgcttcaggcccatattcgtttgggctgttagattgtagcaatggtacaaagatctgcaattgatgtaatttaaccttggttaatggtgtaattttattttattttgttggggtcaaactgaaagcctcaaaggataagagtttttttctgttttttttttttcacacttttttttgttttgttagaattcataaaagggtaatataaacttgttaaaaagaaatacagtagatacgtcatatatacagaaaaagtaagagagacagatgttatcaaataggggggagtaaaacccactctaatactacttgttatgcttaaatataaaaatgaataaaagacttggaaatcaaataaggaagtgagtgattcccacgtcgcaccttcctcctaacccttttcttcatcgccactcaatctttctctctgcaacatcctctcttgtaacgtgtaacttgccaaccttttcttttttattaacccaagttcgttgccggccattaccaattcctttatcccataatctcttctctgcttccttcttccatcacattccccaaaaagtctcaacacagagcgagaagaagactcaggttttgcgagaggagagatagagagacttagaaagctcggtaagtccgaactttggtggttcggcggctctacgtcgtctgtaggtcgagtcgtcctggagctcgcccgttgtttctcgacggcggaggtggaagacaacgaccgaccagccatggacattggcggtccaaccaatatccgccacgtggctcacgttaccttcgatcgcttcgatggcttcctcggccttccctctgagttcgagcctgatgtccccagaaaagctcccagtgcaaggtttatatatactttcttcttcttcttctttaaactcattgagaattggttttgagagatttagatttcatatcatcatcttgtttgtgtttggcagtgcaacggtgtttggggtatcgacagagtcaatgcagttatcatacgactcgagagggaactgtgttcctgtgatactgttgctattgcagagccggctttatgatcaaggaggcttgcaggcagaaggagtattcagaatcactggagagaacagcgaggaagagtttgttagggagcagcttaataaagggatcatacctgatgggattgatgtccattgtttagccggtcttattaaggttcttgttgtgattgtgaatcaccttacctttgtttttgaaaatggtaggcctgagtttgattatgtatgtgtgtgtgggttcaggcttggttcagagagctgccgagaggggtactggatcctctgccatcggagcaagtgatgcagtgtgagtcagatgaggattttgtcaaagttgtgagacttttacctcaaacagaagcttctcttctcaattgggccatcaatctcatggctgatgttattcagtttgagcatgttaataagatgaactctcgtaaccttgctttagtctttgcccccaacatgtctcaggtcagtctttttttattaccaacactaaaatgcacaagttacactctcattaagcatgtgtttgattaatggctgcaataaatcaccgtatagagagctaatatgacagttaaatagcagagcatgattctacaacttcggaggtttgaaatgtattggctctgttttggtttttgtgtgtagatggcagaccctttaactgcattgatgtatgcggtccaagtgatgaagttgctcaagagcctcacagagaagactgtgagagaaagggaagcttcctcttctgtggttgacagaagatgtagcaaagaagccgaagatggcgagaaagaaaaagacaatgaagaggaagaagaagatgaagaagaggaggaagaagaagaagatgaagatgaagatgaagaagaagaaggagatggtgtgtacataattaaggaggaagaagcatcagagataataaaagtggttgctgatgaacacaagtcaggaagcataaagagtgagtttgagggatctagtgctacggattcaaagggagataatggagttgtgcagccacccatttgcagctcgaacccgtaggaagcaagtgaagttgatgctttttttctttttagtttactgtgtgttttggattgctttatttgattgcataatgcatatgtaacttctaagtgatgtagtttgttgttgaatggtgatatgaaatgaagactaactctgtgggatgaacaacataatgtatctctgcttttcatatctagtaagatcatttccattccttaaaagaaacatgaaagagttaacaactaaaatcaaaaagatactaatacagagatgagtcgcatgatgaaaggaagaggaccctttagttcttgggagttattcaagcttgacagaacgatagatgtggcgaataaacatcaaagaagccaagaaacttatggttccaagaaccaagaagagggcgtaacataacaaagcagtgtatcctaagtaaaagctcagctgcagaaatccagtcatgtctgacctgagatagaacaatactccatacccatacatgaacaccgctgtgaatc

SEQ ID NO: 10 - protein, 404 aa, Arabidopsis thaliana
  RHO-like small G-protein of plants (Rop) GTPase activating protein (GAP) 5 (RopGAP5)
MGHILIWFASGPYSFGLLDCSNGFARGEIERLRKLGKSELWWFGGSTSSVGRVVLELARCFSTAEVEDNDRPAMDIGGPTNIRHVAHVTFDRFDGFLGLPSEFEPDVPRKAPSASATVFGVSTESMQLSYDSRGNCVPVILLLLQSRLYDQGGLQAEGVFRITGENSEEEFVREQLNKGIIPDGIDVHCLAGLIKAWFRELPRGVLDPLPSEQVMQCESDEDFVKVVRLLPQTEASLLNWAINLMADVIQFEHVNKMNSRNLALVFAPNMSQMADPLTALMYAVQVMKLLKSLTEKTVREREASSSVVDRRCSKEAEDGEKEKDNEEEEEDEEEEEEEEDEDEDEEEEGDGVYIIKEEEASEIIKVVADEHKSGSIKSEFEGSSATDSKGDNGVVQPPICSSNP

SEQ ID NO: 11 - DNA, 3533 bp, Arabidopsis thaliana
  RHO-like small G-protein of plants (Rop) GTPase activating protein (GAP) 6 (RopGAP6)
aactacaagagtttagatccagctatatatataatgggtttaaacaatgaattctgatcttaaaatagttatcgatttaattaggattaagttaagtaaagaaggaataatatggaggagaagaaggggacaagcgaggaggcaagctaatgcacatgcaagagaacagttttgtctcttctaaatctcaccataaggttacagcagagagatcattgaaacactaaaactaatactctcttttaaattctcttgtttcattaattaacacgttcgtgtgtgtcagttccaacttaacctcggtacgaccaaagatggccacatttcttgtttagaaaaccacatcaattaaaagtgtttaggaaattggcaaagatgaccattgaaatgatttttgccacgattaatttccattttttagttgcaatttggtttatgaaattatattgttaattttttatttttttgatcaaactgtaaacttcattgctagaaattaaatggtttttacatcagagaagtccaaagaaaacagagcctctttggctaaagcatccgctgagagatttggtaaacgaaaaatacaacagaatttgaaagatagaagtgaacgacacaaacaggtgatgtcatgaagacatctaacagaatttctttatataggccaatttaaaagtgtttggcaatttctttattgcgtataaaggcaactaggtacgttactcgtatttcggaggtgcttaaagtaaactagggtcaagtcagtgaccttgatacttcgtttttatgagaaatgttgatgtccaacattgtaatgatataaagtcctacaatcttcagcataaagatctatgtaaggctcgtgttctatagtatgaacacctagacagtttgtttcgaagaaaattggatcatgatcaaagaattgcaaatgaatgagtagactatataaactagtcatagggtacccgaggattcgaattctatatcaacaagctcatcactcacgaggagcggacttaacttgtttggactcaacagcgttgtgattctttaaatatcctaatatcatgtgattttttaaaatgtcttccaaattgtttagaagcaaactatgtacttgtgtaaattaaacgcgttagaaacatatgttatcgatatcaatttctagttcaagataaataaattgtagaaccgtggttggaatcgtatcatcagaaggaaaaagaaacatagatacattaataagtctttcaagtagtgattcccacgtcctacattactctttaaccatttttcttcaccgccacacaatttctccacaacatcttgtgaacgtgtaaacccgtttcgtcgctggcaatgaaaatcacattcccatcccttgcttattacgcattgacattgccgagtaaataccccaatcagaagagaagaaggaagaagaagaagaaagtgtgtttagaattttgggtgacgtggctgtgaaggagagatagcgcgagtgagaagacgcggaaagcccgaactctcgcggttcggcagctccgcgtcagttggtcgaatcgtcttcgagctatctcgttgcttttcgagagatataatggcggaggaaaacgaacggcatgccatggacattagtcgcccaaccaatatctctcatgtggctcatgtaacatacgatcgcttcgatggttttcttggtcttccttctgaattcgagcctgacgtacccaagaagccccctagtgcaaggttcagcttcttcttctttctaatgtaaaccgtgaattgggttgaaacctttgccatgattatttttgggtttgtgtttgcatttgaagtttaagattattatgacattgtaaactgcaaaagaaaatgcaatgctagttttgacattcaaaacaattagaaattctatcacaataatcctaaaagggattcaaaacgttttctaattggaaagatgatcagaaatccatttaacattgactatataaattgtaatctaatgtagtatacaagagacataaccatgaacctttgagaacaatgttttggatatattatgttctaagtgacgttcttgcttcatgatttgttgatctgttttcgggtttgcagtgcaacagtgtttggggtttctacagagtcaatgcagttatcatacgactcgagagggaattgtgttcctaccatactcacgctattacaaagccggctatacgatcaaggaggtttgcaggtagaaggaattttcagaatcacaggggataacagtgaggaggagtttattagagaagagttaaacaaaggagtcctaccagaaggcatcgatatccactgtctcgccggacttataaaggcatggttcagagagttaccgaaaggggtactcgattctctaccatctcaacaagtgatgcagtgtgagtcaggggaggattttgtgaaggttgtgagactcttgtagagtttgaagttgtcaacaagatgacctcccgtaatctagctttagtctttgcacccaacatgtcccaggtcacttcttgcaccacatcacaattttccaagagtttcctttttttctctactcacattgatcttacacagataatgtcagttttatcttagtgctaataccagaatgcatcaatatatcaagcctctgatccgcatactgggtctgttttggttttactgcagatggcagatcctttaacagcattaatgtatgcagtgcaagtgatgaacttgctgaggaacctcacagacaaaacactcagagaaaggaaaattgcgacttcaaatgtagatccttgtgataacagaagtgaagctgaagatggtaatgtcgaagaatataatcaagaagtagaaatatatgttcttgaagaagtagaggaagaagaaggagaagatgttgatgatctggacaacgaagaatccgagagaataacactgcttgctgatgaacacaaaccatcaagcgcagtgaatgctaatgatcgaaagaaacaagaaacatgaagacagccgttggagttggaaacgtcgttagcagtattatgtgtttcgattgatttctctttttcgttatcgaaggtgcatgtaacttaggctaattgttccaaatacaacttatgcattagtgtttatataaacgtatcgaatttttttccatatgacaacctagaaagatgaaagtgccagaacaaattttgagatgtattaaactttttctggtcttttatttttgggcttacttaccctttaaggttaagccagtaataattgttgttaatggatttccccagtgtgatcttaaacaaaagatcaaagcttccaacagcaaaagatttagagattaccatccatagtaagaagcaccttgtacaaatctggacgacggtcacggaacactccccagctttgccttttcgacttgatcatatcaagatcaaactgtgctacaagaacagcttccgatttatcatctgcctcggccacaatttcacctgttggtcctg

SEQ ID NO: 12 - protein, 368 aa, Arabidopsis thaliana
  RHO-like small G-protein of plants (Rop) GTPase activating protein (GAP) 6 (RopGAP6)
MVFTSEKSKENRASLAKASAERFGKRKIQQNLKDRSERHKQIARVRRRGKPELSRFGSSASVGRIVFELSRCFSRDIMAEENERHAMDISRPTNISHVAHVTYDRFDGFLGLPSEFEPDVPKKPPSASATVFGVSTESMQLSYDSRGNCVPTILTLLQSRLYDQGGLQVEGIFRITGDNSEEEFIREELNKGVLPEGIDIHCLAGLIKAWFRELPKGVLDSLPSQQVMQCESGEDFVKVFEVVNKMTSRNLALVFAPNMSQMADPLTALMYAVQVMNLLRNLTDKTLRERKIATSNVDPCDNRSEAEDGNVEEYNQEVEIYVLEEVEEEEGEDVDDLDNEESERITLLADEHKPSSAVNANDRKKQET

SEQ ID NO: 13 - protein, 27 aa, Artificial Sequence
  Description of Artificial Sequencedominant negative RopGAP polypeptide, Cdc42/Rac-interactive binding (CRIB) motif of RopGAP4
MEIGWPTDVRHVAHVTFDRFHGFLGLP

SEQ ID NO: 14 - protein, 40 aa, Artificial Sequence
  Description of Artificial Sequencedominant negative RopGAP polypeptide
LNKMNARNIAMVFAPNMTQMLDPLTALMYAVQVMNFLKTL

SEQ ID NO: 15 - DNA, 10 bp, Artificial Sequence
  Description of Artificial Sequenceantioxidant response element (ARE) consensus sequence
gtgacawwgc

SEQ ID NO: 16 - protein, 27 aa, Artificial Sequence
  Description of Artificial SequenceCdc42/Rac-interactive binding (CRIB) motif in RopGAP1
MEIGWPTNVRHVAHVTFDRNGGFLGLP

SEQ ID NO: 17 - protein, 25 aa, Artificial Sequence
  Description of Artificial Sequenceconsensus motif for RopGAP CRIB motifs
IGXXTXVXHXXHVTFDRXXGFXGLP

SEQ ID NO: 18 - protein, 220 aa, Artificial Sequence
  Description of Artificial SequenceCRIB and GAP region of RopGAP4
MEIGVPTDVRHVAHVTFDRFHGFLGLPVEFEPEVPRRAPSASATVFGVSTESMQLSYDTRGNIVPTILLMMQSHLYSRGGLRVEGIFRINGENGQEEYIREELNKGIIPDNIDVHCLASLIKAWFRELPSGVLDSLSPEQVMESESEDECVELVRLLPSTEASLLDWAINLMADVVEMEQLNKMNARNIAMVFAPNMTQMLDPLTALMYAVQVMNFLKTL

SEQ ID NO: 19 - DNA, 20 bp, Artificial Sequence
  Description of Artificial Sequence ROPGAP4-forward PCR primer
agagggaacatcgtgcctac

SEQ ID NO: 20 - DNA, 20 bp, Artificial Sequence
  Description of Artificial Sequence ROPGAP4-reverse PCR primer
aactgtctactgctgctctg

SEQ ID NO: 21 - DNA, 20 bp, Artificial Sequence
  Description of Artificial Sequence ADH-forward PCR primer
tctactgggttaggagcaac

SEQ ID NO: 22 - DNA, 20 bp, Artificial Sequence
  Description of Artificial Sequence ADH-reverse PCR primer
ttgatttccgagaatggcac

SEQ ID NO: 23 - DNA, 16 bp, Artificial Sequence
  Description of Artificial Sequence ACT2-forward PCR primer
aaaaatggccgatggt

SEQ ID NO: 24 - DNA, 21 bp, Artificial Sequence
  Description of Artificial Sequence ACT2-reverse PCR primer
ctggttcgtggtggtgagttt